(19) United States
(12) Patent Application Publication
(10) Pub. No.: US 2022/0193185 A1
(43) Pub. Date: Jun. 23, 2022

(54) COMBINATION OF SYNTHETIC PEPTIDES WITH AFFINITY TO THE TGF-ß RECEPTOR AND WITH AFFINITY TO THE IL-10 RECEPTOR, PHARMACEUTICAL COMPOSITION AND THEIR USE AS IMMUNOMODULATORS IN THE TREATMENT OF AUTOIMMUNE, INFLAMMATORY OR ALLERGIC DISEASES

(71) Applicants: Universidade Federal de Uberlândia, Uberlândia (BR); Cirino Alberto Goulart EIRELI EPP (Biogenetics), Uberlândia (BR)

(72) Inventors: Luiz Ricardo Goulart FILHO, Uberlândia (BR); Fatima D. FERREIRA-BRIZA, Salzburg (AT); Emilia Rezende VAZ, Uberlândia (BR); Galber Rodrigues ARAUJO, Salzburg (AT); Carlos Ueira VIEIRA, Uberlândia (BR); Lorenz AGLAS, Salzburg (AT)

(21) Appl. No.: 17/601,628

(22) PCT Filed: Apr. 6, 2020
(86) PCT No.: PCT/BR2020/050118

§ 371 Date: Oct. 5, 2021

(30) Foreign Application Priority Data
Apr. 5, 2019 (BR) 10 2019 007048 0

(51) Int. Cl. A61K 38/10; A61K 39/35; A61P 29/00; A61P 37/08

(52) U.S. Cl. CPC A61K 38/10; A61P 37/08; A61P 29/00; A61K 39/35

(57) ABSTRACT

The invention concerns the selection and characterization of synthetic peptides, which have affinity for receptors present in cells of the immune system, more particularly peptides with affinity for the TGF-β receptor and peptides with affinity for the IL-10 receptor, as well as pharmaceutical compositions and the use of such combined peptides to prepare drugs or immunogenic compositions. Said synthetic peptides can bind to cell receptors and promote an important regulatory profile for the treatment and/or prophylaxis of diseases, more particularly chronic or acute inflammatory and/or allergic diseases.

### FIELD OF THE INVENTION

The invention concerns the combination of synthetic peptides that have affinity for different receptors present in cells of the immune system, more particularly the combination of synthetic peptides with affinity to the transforming growth factor beta (from the acronym in English: TGF-β) receptor, with synthetic peptides with affinity to the interleukin-10 (IL-10) receptor. Both receptors are known to play an important role in modulating immune, inflammatory, and allergic responses. In addition, the present invention also describes pharmaceutical compositions comprising the combination of at least one of each of these synthesized peptides and the use of said peptides to prepare drugs or immunogenic compositions for the treatment and/or prophylaxis of diseases in which there is an immunological disorder, more particularly in which the diseases related to the immune disorder are: chronic or acute inflammatory disease, allergic and/or autoimmune diseases.

### BACKGROUND OF THE INVENTION

Transforming growth factor (TGF)-β is a pleiotropic cytokine that acts on several biological processes, including embryonic development and cell proliferation, differentiation, adhesion, migration, and apoptosis. Its signaling has been proposed to control autoreactive peripheral T cells, present in patients with autoimmune diseases. Said cytokine also plays an important role in inducing auxiliary T cells capable of regulating cytokine production and pathway activation in order to repair the immune response.

TGF-β1 is produced by several cell types, such as platelets, neutrophils, malignant cells, dendritic cells, and macrophages. As a result, such cytokine has an important effect on inflammatory, allergic, and autoimmune diseases. Due to the diversity of TGF-β1, many factors are responsible for activating canonical or non-canonical pathways to induce multiple biological effects and interfere with different pro-inflammatory pathways. This cytokine can modulate different pathways directly related to TGF-β, receptors, as well as in response to crosstalk signaling.

TGF-β1 is a cytokine that plays an important role in allergic inflammation. It can suppress effector T cells and is involved in the development of conventional, innate, regulatory (Treg) T cells. T cells play a key role in maintaining peripheral tolerance against commensal, autologous, and environmental antigens. In mice with the TGF-β1 gene disrupted, and in mice without the (TORbRII) receptor II, severe inflammatory responses, tissue necrosis and early death were observed, confirming the regulatory role of TGF-β in peripheral tolerance.

IL-10 is a regulatory pleiotropic cytokine produced by different types of cells: monocytes, T cells, macrophages, dendritic cells (DC), natural killer cells (NK) and B cells. Since it influences different cell types, IL-10 has been shown to be an important molecule associated with a wide range of diseases, disorders, and conditions, including inflammatory and allergic conditions, immunity-related disorders, and cancer, due to its ability to regulate the humoral and cellular immune response. Thus, understanding how IL-10 regulates different cell types is crucial for the development of new intervention strategies for the treatment of various pathologies.

T lymphocytes are among the main factors involved in regulating the adaptive immune response in inflammatory diseases. In a healthy immune system, Type I helper T cells (Th1) are required for the elimination of many intracellular pathogens, whereas Th2 cells are required for the elimination of helminth infection. A disruption in the Th1/Th2 balance can lead to immunological disturbances with fatal outcomes. In an unbalanced immune system, Th1 cells are involved in delayed hypersensitivity immune reactions, while Th2 cells are involved in allergic diseases. Th1 lymphocytes produce IFN-g and IL-2 to support cell-mediated immunity, while Th2 lymphocytes produce cytokines such as IL-4, IL-5, and IL-13, which play important roles in exacerbating the allergic response. IL-4 is the main cytokine for the polarization of a Th2 response, which, together with IL-13, induces the class change of immunoglobulins production from B lymphocytes to IgE. Allergic reactions are initiated after subsequent exposure to the allergen, in which the allergen binds to specific IgE along with the allergen bound to the high-affinity FIERI receptor on mast cells or basophils, triggering cell degranulation. This event alerts the immune system to local infection and propagates the allergic inflammatory response. Thus, the modulation of cell cytokines found in a Th1/Th2 profile is essential for the maintenance or restoration of immune homeostasis.

Birch pollen (Verrucous birch) is the main cause of pollinosis in the temperate climate zone of the northern hemisphere. The vast majority of patients allergic to birch (over 90%) react to its main allergen, Bet v 1, which has the ability to promote the induction of a Th2 profile, and is used as a marker of allergy to birch pollen. Allergen-specific immunotherapy (ASIT) modulates the natural course of allergies, leading to improvement or even complete remission of allergic symptoms (Mobs, C. et al. Cellular and humoral mechanisms of immune tolerance in immediate-type allergy induced by specific immunotherapy. Int Arch Allergy Immunol. 2008; 147(3):171-8). However, although ASIT is the treatment of choice for many patients, local and systemic allergic side effects have been reported. Sublingual allergen immunotherapy has also been associated with side effects.

Transforming growth factor b1 (TGFβ) is a cytokine that has important immunoregulatory properties, as reflected by its ability to suppress Th1 and Th2 responses and by inducing the expression of transcription factor fork box protein 3 (Foxp3). Foxp3 is a master regulator involved in the development and function of regulatory T cells (Treg). Treg cells are essential for the maintenance of immune homeostasis and the induction of immune tolerance. Its dysfunction can lead to the development of autoimmune diseases, immunopathology, and allergy. On the other hand, Interleukin 10 (IL-10) is a cytokine associated with important immunoregulatory functions, including the survival and proliferation of several cells of the immune system. Thus, peptides that can mimic the activation domain of TGF-β1 and IL-10 may be promising candidates for suppressing the Th2 response.

To develop new molecules with pharmaceutical approaches, the Phage Display (PD) methodology may be a good option, as it allows us to select small molecules that can imitate specific amino acids from large proteins and induce the same or better response as expected with the natural molecule or in comparison with other recombinant molecules already described. Peptides selected by PD have low toxicity and high receptor/protein affinity.

Therapeutic mimetic peptides are potential drug candidates for many different diseases, as they have the potential to antagonize, stimulate, increase, or modulate the activity of natural ligands, binding to specific cell surface receptors and triggering intracellular pathways.

With the purpose of seeking therapeutic strategies, patent WO2012167143 A1 reports the use of the phage display technology to obtain a specific antibody binding to the TGF-β molecule that can be used in patients who have disorders related to TGF-β expression, such as cancer. Such results show the efficiency of the technique in the development of recombinant antibodies in the treatment of diseases.

Patent application BR 102015003903-4 describes 27 synthetic peptides and their inverse sequences, selected by Phage Display, which have affinity for the TGF-β receptor. Then, said patent application describes the use of such peptide sequences synthesized in pharmaceutical compositions in the regulation of the immune system, in order to obtain the inflammation control or allow the treatment of inflammatory and autoimmune diseases. Said patent application also describes the possibility of using such synthesized peptides in methods of diagnosis or prognosis of related diseases.

Recombinant IL-10, for example, is already shown to be safe, well tolerated, and effective in patients with Crohn's disease (Fedorak, R. N. et al. Gastroenterology 2000 December; 119 (6): 1473-82). Patent application BR 102017010787-6 results in 101 synthetic peptides, selected by Phage Display, which have affinity for the IL-10 receptor. Therefore, the aforementioned patent application shows knowledge of the use of these synthesized peptide sequences in the treatment and monitoring of an inflammatory process, allergic diseases and autoimmune diseases.

### Purpose of the Invention

The present patent application aimed to select new mimetic peptides to TGF-β1 and IL-10, by PD technology, which had an improved capacity to modulate and attenuate immune response, particularly to control inflammatory and allergic responses.

Considering the studied peptides, eight (8) peptides with similar action to TGF-β1 were selected and synthesized and four (4) peptides with similar action to IL-10 were selected and synthesized, having their predicted three-dimensional structures.

The data obtained showed the ability of combining TGF-β1 mimetic peptides with IL-10 mimetic peptides to reduce the allergic response and inhibit inflammatory pathways in the assays performed. Such result shows the potential for pharmaceutical use of the combination of synthetic peptides presented here in the modulation of inflammatory allergic responses.

### BRIEF DESCRIPTION OF THE INVENTION

The present patent application describes synthetic peptides with affinity for the TGF-β receptor and synthetic peptides with affinity for the Interleukin-10 (IL-10) receptor. Such peptides were synthesized, and their three-dimensional structures were predicted. All of them were synthesized to present Histidine and Alanine as the first amino acid residue, in some cases Alanine as the second amino acid residue, and a sequence of three Glycine and a Serine as the last four amino acid residues. Such peptides showed important activity on immunological modulatory and regulatory pathways. Therefore, they can be used in the treatment and/or prophylaxis of diseases related to an immune disorder, such as: chronic or acute inflammatory, allergic and/or autoimmune diseases. Therefore, TGF-β1 and IL10 mimetic peptides may be promising candidates for attenuating inflammatory and allergic reactions when used in combination.

Another embodiment of the present application relates to methods of treatment and/or prophylactic of immune disorder related diseases, wherein the immune disorder related diseases are chronic or acute inflammatory, allergic and/or autoimmune diseases, comprising administering in a subject a pharmaceutically effective dose of a combination of at least one of the synthetic peptides mimetic to TGF-β1 and at least one of the synthetic peptides mimetic to IL-10.

Another embodiment of the present application are pharmaceutical compositions comprising a combination of at least one of the synthetic peptides mimetic to TGF-β1 and at least one of the synthetic peptides mimetic to IL-10, plus at least one pharmaceutically acceptable carrier or compound. Pharmaceutical Composition is for use in the treatment and/or prophylaxis of diseases related to an immune disorder, in which the diseases related to an immune disorder are chronic or acute inflammatory, allergic and/or autoimmune diseases.

Another embodiment of the present application is the use of the combination of synthetic peptides or pharmaceutical compositions described herein, for the preparation of a drug or immunogenic composition for use in the treatment and/or prophylaxis of diseases related to an immunological disorder, in which the immune disorder related diseases are chronic or acute inflammatory, allergic and/or autoimmune diseases.

### BRIEF DESCRIPTION OF DRAWINGS

The present invention will be better understood based on the description below, taken in conjunction with the attached figures, wherein:

FIG. 1A shows the predicted three-dimensional structure of Pep 1-TGF-β.

FIG. 1B shows the predicted three-dimensional structure of Pep 2-TGF-β1.

FIG. 1C shows the predicted three-dimensional structure of Pep 3-TGF-β1.

FIG. 1D shows the predicted three-dimensional structure of Pep 4-TGF-β1.

FIG. 1E shows the predicted three-dimensional structure of Pep 5-TGF-β1.

FIG. 1F shows the predicted three-dimensional structure of Pep 6-TGF-β1.

FIG. 1G shows the predicted three-dimensional structure of Pep 7-TGF-β1.

FIG. 1H shows the predicted three-dimensional structure of Pep 8-TGF-β1.

FIG. 2 shows an analysis of the aggregation behavior of mimetic peptides to TGF-β1 synthesized in solution analyzed by DLS.

FIG. 3 shows an analysis of recognition by the anti-TGF-β1 antibody of the TGF-β1 mimetic peptides synthesized on the cell surface of the A549 line.

FIG. 4A shows the activation of the TFGβRII/SMAD-dependent pathway, through the analysis of the production of SEAP (secreted alkaline phosphatase) after treatment with the TGF-β mimetic peptides synthesized described herein for 12 hours.

FIG. 4B shows the activation of the TFGβRII/SMAD-dependent pathway, by analyzing the production of SEAP (secreted alkaline phosphatase) after treatment with the synthesized peptides described herein for 24 hours.

FIG. 4C shows the activation of the TFGβRII/SMAD-dependent pathway, through the analysis of the production of SEAP (secreted alkaline phosphatase) after treatment with the synthesized peptides described herein for 48 hours.

FIG. 5A shows an analysis of the effect of synthesized peptides in the NFkB pathway, after pretreatment with the peptides for 1 hour, followed by stimulation with TNF-α and analyzed after 12 hours.

FIG. 5B shows an analysis of the effect of synthesized peptides in the NFkB pathway, after pretreatment with the peptides for 1 hour, followed by stimulation with TNF-α and analyzed after 24 hours.

FIG. 5C shows an analysis of the effect of synthesized peptides in the NFkB pathway, after pretreatment with the peptides for 1 hour, followed by stimulation with TNF-α and analyzed after 48 hours.

FIG. 5D shows an analysis of the effect of synthesized peptides in the NFkB pathway, after pre-stimulation with TNF-α for 1 hour, followed by the addition of peptides and analyzed after 12 hours.

FIG. 5E shows an analysis of the effect of synthesized peptides in the NFkB pathway, after pre-stimulation with TNF-α for 1 hour, followed by the addition of peptides and analyzed after 24 hours.

FIG. 5E shows an analysis of the effect of synthesized peptides in the NFkB pathway, after pre-stimulation with TNF-α for 1 hour, followed by the addition of peptides and analyzed after 48 hours.

FIG. 6A shows analysis to investigate the action of peptides in the TLR4 pathway, after pretreatment with the synthesized peptides, followed by LPS stimulation and analyzed after 12 hours.

FIG. 6B shows analysis to investigate the action of peptides in the TLR4 pathway, after pre-stimulation with LPS, followed by the addition of peptides and analyzed after 12 hours.

FIG. 7A shows an ELISA assay to assess the binding of rTGF11 and Pep 2 on the cell surface of Jurkat.

FIG. 7B demonstrates a luciferase reporter assay showing IL-8 gene expression in TNF-α stimulated A549 cells and treated with Pep 2.

FIG. 7C shows an IL-8 production/secretion assay in A549 cells stimulated by TNF-α and treated with Pep 2.

FIG. 7D shows an assay of the action of Pep 2 on TNF-α secretion.

FIG. 7E shows an assay of the action of Pep 2 on IL-10 secretion.

FIG. 7F shows a mediator release assay based on rat basophil leukemia (RBL) cells, which possess humanized FIERI receptor to investigate the effect of Pep 2 on IgE-mediated basophil degranulation.

FIG. 8A shows an immunization scheme in animals.

FIG. 8B shows an immunization assay with recombinant Phl p5, showing the action of Pep 2 in basophil degranulation.

FIG. 8C shows an immunization assay with recombinant Phl p5, showing the action of Pep 2 on IgE levels.

FIG. 8D shows an immunization assay with recombinant Phl p5, showing the action of Pep 2 on IgG1 levels.

FIG. 8E shows an immunization assay with recombinant Phl p5, showing the action of Pep 2 on IgG2a levels.

FIG. 8F shows an immunization assay with recombinant Phl p5, showing the action of Pep 2 on IgA levels.

FIG. 9A shows an immunization assay with recombinant Phl p5 in splenocytes, showing the action of Pep 2 on IFN-γ levels.

FIG. 9B shows an immunization assay with recombinant Phl p5 in splenocytes, showing the action of Pep 2 on IL-4 levels.

FIG. 9C shows an immunization assay with recombinant Phl p5 in splenocytes, showing the action of Pep 2 in the induction of IL-10.

FIG. 9D shows a re-immunization assay with recombinant Phl p5 in splenocyte supernatant, showing the action of Pep 2 on IFN-γ levels.

FIG. 9E shows a re-immunization assay with recombinant Phl p5 in splenocyte supernatant, showing the action of Pep 2 on IL-2 levels.

FIG. 9F shows a re-immunization assay with recombinant Phl p5 in splenocyte supernatant, showing the action of Pep 2 on IL-4 levels.

FIG. 9G shows a re-immunization assay with recombinant Phl p5 in splenocyte supernatant, showing the action of Pep 2 on IL-5 levels.

FIG. 9H shows a re-immunization assay with recombinant Phl p5 in splenocyte supernatant, showing the action of Pep 2 on IL-13 levels.

FIG. 9I shows a re-immunization assay with recombinant Phl p5 in splenocyte supernatant, showing the action of Pep 2 in inducing IL-10.

FIG. 10A shows an assay to investigate CD4+CD25+Foxp3+cells that express GATA3 in splenocytes, in mice sensitized with Phl p5 and treated with Pep 2.

FIG. 10B shows an assay to investigate CD4+CD25+Foxp3+cells that express GATA3 in splenocytes, in mice sensitized with Phl p5 and treated with Pep 2.

FIG. 10C shows an assay to investigate CD4+CD25+Foxp3+cells expressing CTLA4 in splenocytes, in mice sensitized with Phl p5 and treated with Pep 2.

FIG. 10D shows an assay to investigate CD4+CD25+Foxp3+cells that express CTLA4 in splenocytes, in mice sensitized with Phl p5 and treated with Pep 2.

FIG. 10E shows an assay to investigate CD4+CD25+Foxp3+cells that express KI67 in splenocytes, in mice sensitized with Phl p5 and treated with Pep 2.

FIG. 10F shows an assay to investigate CD4+CD25+Foxp3+cells that express KI67 in splenocytes, in mice sensitized with Phl p5 and treated with Pep 2.

FIG. 11A shows the analysis of GFP+CD4+ lymphocytes isolated from 4get mice 5 days after immunization.

FIG. 11B shows that the synthesized peptide administered along the pollen extract of grasses suppresses TH2 polarization.

FIG. 11C shows that the synthesized peptide administered along the pollen extract of grasses suppresses the TH2 polarization.

FIG. 12A shows the predicted three-dimensional structure of Pep 1-IL10.

FIG. 12B shows the predicted three-dimensional structure of Pep 2-IL10.

FIG. 12C shows the predicted three-dimensional structure of Pep 3-IL10.

FIG. 12D shows the predicted three-dimensional structure of Pep 4.

FIG. 12E shows the aggregation behavior of peptides Pep 1-IL10, Pep 2-IL10, Pep 3-IL10 and Pep 4-I L10 in solution.

FIG. 13A shows an analysis of NFkB expression after treatment with the synthesized peptides mimetic to IL-10 described herein for 12 hours.

FIG. 13B shows an analysis of NFkB expression after treatment with the IL-10 mimetic synthesized peptides described herein for 24 hours.

FIG. 13C shows an analysis of NFkB expression after treatment with the synthesized peptides mimetic to IL-10 described herein for 48 hours.

FIG. 14A shows an analysis of NFkB expression, using an activating molecule, TNF-α, not only in the control, but after a pretreatment with the synthesized peptides mimetic to IL-10 for 12 hours.

FIG. 14B shows an analysis of NFkB expression, using an activating molecule, TNF-α, not only in the control, but after a pretreatment with the synthesized peptides mimetic to IL-10 for 24 hours.

FIG. 14C shows an analysis of NFkB expression, using an activating molecule, TNF-α, not only in the control, but after a pretreatment with the synthesized peptides mimetic to IL-10 for 48 hours.

FIG. 15A shows an analysis of NFkB expression when cells were pretreated with TNF-α and the synthesized peptides mimetic to IL-10 were added later, after treating the cells for 12 hours.

FIG. 15B shows an analysis of NFkB expression, when cells were pretreated with TNF-α and the synthesized peptides mimetic to IL-10 were added later, after treating the cells for 24 hours.

FIG. 15C shows an analysis of NFkB expression, when cells were pretreated with TNF-α and the synthesized peptides mimetic to IL-10 were added later, after treatment of cells for 48 hours.

FIG. 16A shows an analysis of the IFN signaling pathway after treatment with the synthesized peptides mimetic to IL-10 described herein for 12 hours.

FIG. 16B shows an analysis of the IFN signaling pathway after treatment with the synthesized peptides mimetic to IL-10 described herein for 24 hours.

FIG. 16C shows an analysis of the IFN signaling pathway after treatment with the synthesized peptides mimetic to IL-10 described herein for 48 hours.

FIG. 17A shows an analysis of the IFN signaling pathway, when cells were pretreated with IFN and the synthesized peptides mimetic to IL-10 were added later, after treating the cells for 12 hours.

FIG. 17B shows an analysis of the IFN signaling pathway, when cells were pretreated with IFN and the synthesized peptides mimetic to IL-10 were added later, after treating the cells for 24 hours.

FIG. 17C shows an analysis of the IFN signaling pathway, when cells were pretreated with IFN and the synthesized peptides mimetic to IL-10 were added later, after treating the cells for 48 hours.

FIG. 18A shows the ability of the synthesized peptides mimetic to IL-10 to reduce the activation of the TLR4 signaling pathway after 12 hours of treatment with the synthesized peptides.

FIG. 18B shows the ability of the synthesized peptides mimetic to IL-10 to reduce the activation of the TLR4 signaling pathway when cells were pretreated with lipopolysaccharides (LPS) and the synthesized peptides were added later, after 12 hours of treatment with the peptides.

FIG. 19A shows the effect of treatment with the synthesized peptides mimetic to IL-10 in reducing cell activation in CD11c+CD86 cells, after 24 hours of treatment.

FIG. 19B shows the effect of treatment with the synthesized peptides mimetic to IL-10 in reducing cell activation in CD11c+CD40+cells, after 24 hours of treatment.

FIG. 20 shows the ability of synthesized peptides mimetic to IL-10 (Pep 1-IL-10) in decreasing the activation of dendritic cells after 24 hours of treatment.

FIG. 21A shows the effect of synthesized peptides mimetic to IL-10 (Pep 1-IL-10) on mediated IgE release.

FIG. 21B shows the effect of rlL10 on mediated IgE release.

FIG. 23A shows the immunization scheme of animals with combination of peptides.

FIG. 23B shows the titration curve for the mediator release assay performed with sera from all groups showing that mice treated with the combination of synthesized peptides had substantially lower levels of basophil degranulation.

FIG. 23C shows the mediator release assay performed with individual sera reinforcing the action of the combination of synthesized peptides in the downregulation of IgE-specific basophil degranulation.

FIG. 24A shows the levels of IFN-γ produced by splenocytes restimulated with Bet v 1 and the combination of synthesized peptides.

FIG. 24B shows the levels of IL-4 produced by splenocytes restimulated with Bet v 1 and the combination of synthesized peptides.

FIG. 24C shows the levels of IL-10 (C) produced by splenocytes restimulated with Bet v 1 and the combination of synthesized peptides.

FIG. 25A shows the ability of the combination of synthesized peptides to modulate the production of cytokine IL-2 in splenocytes.

FIG. 25B shows the ability of the combination of synthesized peptides to modulate the production of cytokine IL-4 in splenocytes.

FIG. 25C shows the ability of the combination of synthesized peptides to modulate the production of the cytokine IL-5 in splenocytes.

FIG. 25D shows the ability of the combination of synthesized peptides to modulate the production of the cytokine IL-13 in splenocytes.

FIG. 26 shows the ability of the combination of synthesized peptides to modulate the degranulation of basophils.

### DETAILED DESCRIPTION OF THE INVENTION

Synthetic Peptides with Affinity for the TGF-β Receptor

The present patent application describes 8 synthetic peptides with affinity to the TGF-β receptor, which comprise or consist of the amino acid sequences: peptide 1-TGF-β1 (Pep 1-TGF-β1): SEQ ID N 01; peptide 2-TGF-β1 (Pep 2-TGF-β1): SEQ ID N 02; peptide 3-TGF-β1 (Pep 3-TGF-β1) SEQ ID N 03; peptide 4-TGF-β1 (Pep 4-TGF-β1) SEQ ID N 04; peptide 5-TGF-β1 (Pep 5-TGF-β1) SEQ ID N 05; peptide 6-TGF-β1 (Pep 6-TGF-β1) SEQ ID N 06; peptide 7-TGF-β1 (Pep 7-TGF-β1) SEQ ID N 07; and peptide 8-TGF-β1 (Pep 8-TGF-β1) SEQ ID N 08. Such peptides showed important activity on iμMunological modulatory and regulatory pathways. Therefore, they can be used in the treatment and/or prophylaxis of diseases related to an immune disorder, such as: chronic or acute inflammatory, allergic and/or autoimmune diseases.

The peptides described in this patent application were selected using the Phage Display (PD) methodology. The selection was performed by evaluating the ability of said peptides to recognize the TGF-β1 receptor on A549 cells, using an ELISA assay. MaxiSorp 96-well microtiter plates were coated with 1×106 cells diluted in PBS and incubated overnight at 4° C. Cells were blocked with 3% PBS-BSA for 1 h at 37° C., washed once with PBS and incubated with pg/ml of each TGF-β1 mimetic peptide or 5 ng/ml of recombinant TGF-β1 for 1 h at 37° C. After washing, the plate was incubated for 1 h at 37° C. with anti-TGF-β1 antibody. The plate was washed and incubated with HRP-labeled anti-human IgG antibody for 1 h at 37° C. After washing, TMB substrate solution was added to the plate; the reaction was stopped by adding the 2N H2SO4 solution and read at 492 nm in a microplate reader.

Peptides with high affinity for cellular receptors were then chemically synthesized following the phage selection manual (Barbas, C F et al. Phage Display: a laboratory manual. Cold Spring Harbor Laboratory Press (2001)) and all of them were synthesized in a similar way so as to present Histidine and Alanine as the first and second amino acid residues, respectively, and a sequence of three Glycine and a Serine as the last four amino acid residues, as well as some peptides were synthesized with an identity or similarity of at least 85% between them, with the replacement, for example, of serine residues by cysteine. Such specific substitution modified the three-dimensional structure, inducing the formation and conformation of disulfide bridges, or not. Its three-dimensional structures were predicted and demonstrated herein by the figures as follows.

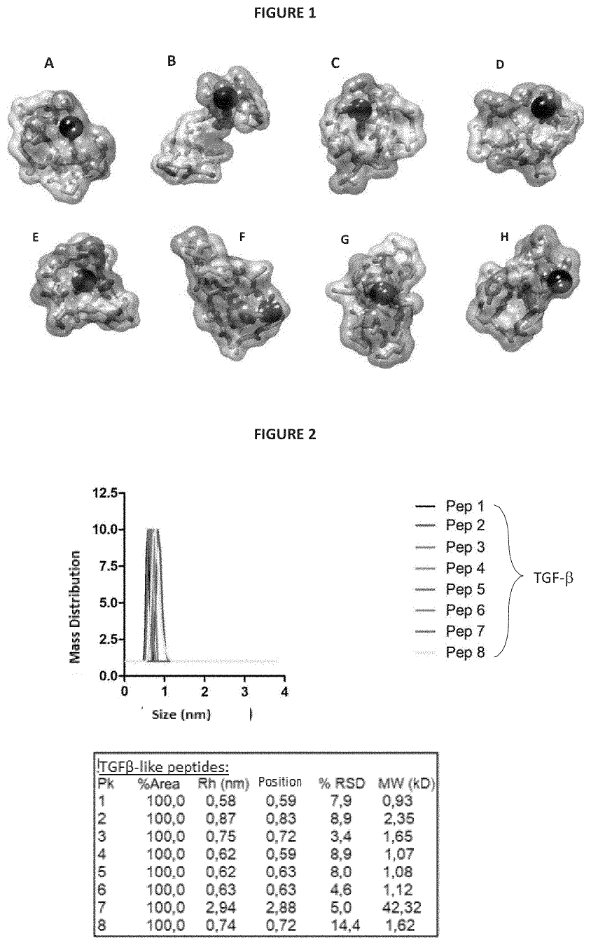

Peptide 1-TGF-β1 (Pep 1-TGF-β1), SEQ ID N 01, was synthesized by inducing the formation of disulfide bridges between cystine residues, and has its three-dimensional structure predicted in FIG. 1A.

Peptide 2-TGF-β1 (Pep 2-TGF-β1), SEQ ID N 02, was synthesized by inducing the formation of disulfide bridges between cystine residues, and has its three-dimensional structure predicted in FIG. 1B.

Peptide 3-TGF-β1 (Pep 3-TGF-β1), SEQ ID N 03, is free of disulfide bridges and has its three-dimensional structure predicted in FIG. 1C.

Peptide 4-TGF-β1 (Pep 4-TGF-β1), SEQ ID N 04, was synthesized by inducing the formation of disulfide bridges between cystine residues and has its three-dimensional structure predicted in FIG. 1D.

Peptide 5-TGF-β1 (Pep 5-TGF-β1), SEQ ID N 05, is free of disulfide bridges and has its three-dimensional structure predicted in FIG. 1E.

Peptide 6-TGF-β1 (Pep 6-TGF-β1), SEQ ID N 06, is free of disulfide bridges and has its three-dimensional structure predicted in FIG. 1F.

Peptide 7-TGF-β1 (Pep 7-TGF-β1), SEQ ID N 07, is free of disulfide bridges and has its three-dimensional structure predicted in FIG. 1G.

Peptide 8-TGF-β1 (Pep 8-TGF-β1), SEQ ID N 08, is free of disulfide bridges and has its three-dimensional structure predicted in FIG. 1H.

Therefore, the synthesized peptides comprising or consisting of one of SEQ IDs No. 01, 02 or 04 may present three-dimensional formation with disulfide bridges. The synthesized peptides comprising or consisting of one of the SEQ IDs Nos. 03, 05, 06, 07 and 08 have linearized three-dimensional conformation, free from disulfide bridges. (FIGS. 1A-H).

In addition, Pep 1 and Pep 5 peptides; Pep 2 and Pep 3 peptides; and Pep 4 and Pep 6 peptides; were synthesized with an identity or similarity of at least 85% between them, respectively. The amino acid residues at positions three (3) and eleven (11) were substituted from Serine in peptides Pep 3, Pep 5, and Pep 6, to Cysteine in peptides Pep 1, Pep 2 and Pep 4.

Peptide structures were predicted according to each modification made (FIG. 1A-H). The black sphere represents the first residue (C-alpha atom) in each peptide, while the blue and red clusters indicate hydrophilic and hydrophobic regions, respectively.

Although the peptides have different structures, the DLS result shows that all peptides are found as monomers with hydrodynamic radius between 0.58 nm (Pep1) and 2.94 nm (Pep7) (FIG. 2).

Once the selected peptides were synthesized, several assays were carried out to determine the activity of such peptides. It is noteworthy here that both in the examples that we will demonstrate below and in the figures attached here, all peptides in general and in different concentrations showed surprising and positive results in modulating the immune, inflammatory and allergic response. However, we will highlight here, throughout the description, just as an example, those results that were statistically more significant. This does not exclude any other results that have not been expressly mentioned in the examples throughout the descriptive report, but which can be directly verified in the figures.

### Example 1—Recognition of Anti-TGF-β1 Antibody on the Cell Surface of the A549 Line

To investigate whether the synthesized peptides could bind to cell receptors, an ELISA assay was performed on cells of the A549 line using commercial anti-TGF-β1 antibody.

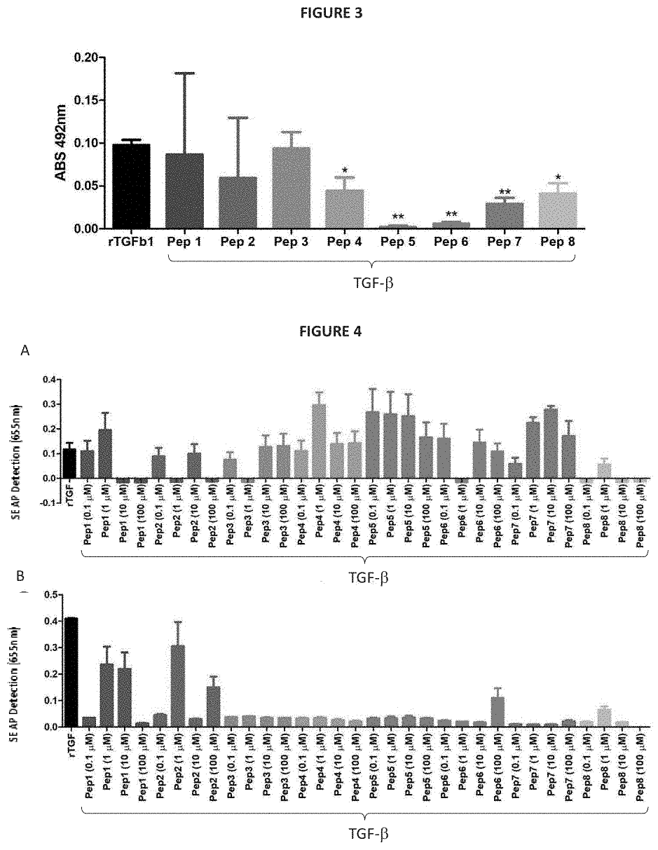

All eight tested peptides were able to recognize and bind to receptors on the cell surface of A549 cells. The synthesized peptides Pep 1-TGF-β1, Pep 2-TGF-β1 and Pep 3-TGF-β1, bound to cell receptors, were recognized by the anti-TGF-β1 antibody, in the same way that the antibody recognized the TGF-β1 recombinant molecule. However, the peptides Pep 4-TGF-β1, Pep 5-TGF-β1, Pep 6-TGF-β1, Pep 7-TGF-β1 and Pep 8-TGF-β1, also bound to cell receptors, were recognized as statistically different by the antibody, compared to recombinant TGF-β1 [Pep 4 (P<0.05), Pep 5 (P<0.005), Pep 6 (P<0.005), Pep 7 (P<0.005) or Pep 8 (P<0.00<0.05)] (FIG. 3).

### Example 2—Ability of TGF-β1 Mimetic Peptides to Activate the SMAD-Dependent Pathway

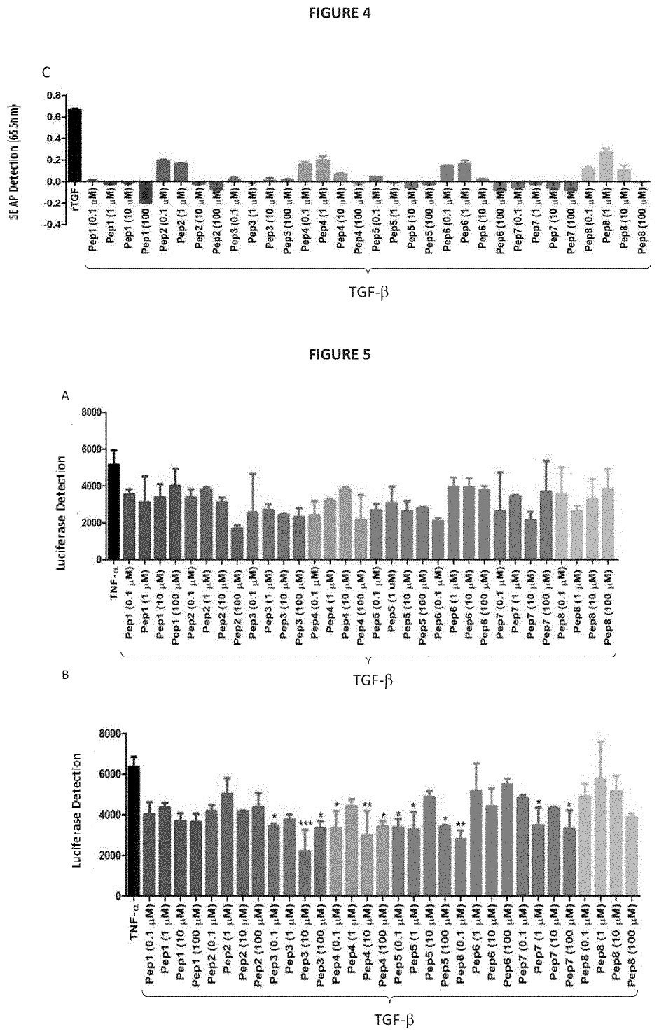

To investigate the ability of peptides to bind TFGβRII and activate the SMAD-dependent pathway, HEK-blue-TGFβ cells were treated with peptides at 0.1 μM, 1 μM, 10 μM and 100 μM for 12 hours (FIG. 4A), 24 hours (FIG. 4B) and 48 hours (FIG. 4C). After 12 hours of stimulation (FIG. 4A), all peptides were able to bind to the receptor. Particularly for some peptides, and at specific concentrations, we obtained statistically significant results such as: Pep 1 (0.1 μM and 1 μM), Pep 2 (0.1 μM and 10 μM), Pep 3 (0.1 μM, 10 μM and 100 μM), Pep 4 (0.1 μM, 1 μM; 10 μM and 100 μM) Pep 5 (0.1 μM, 1 μM, 10 μM and 100 μM), Pep 6 (0.1 μM, 10 μM and 100 μM) and Pep 7 ((0.1 μM: 1 μM; 10 μM and 100 μM) and Pep 8 (1 μM). However, when cells were analyzed after 24 hours (FIG. 4B), activation was maintained, with significant result, by Pep 1 (1 μM and 10 μM), Pep 2 (1 μM and 100 μM), Pep 6 (100 μM) and Pep 8 (1 μM). After 48 hours (FIG. 4B) the activation was maintained, with significant results, at least for Pep 2 (0.1 μM and 1 μM), Pep 4 (0.1 μM and 1 μM), Pep 6 (0.1 μM and 1 μM) and Pep 8 (0.1 μM, 1 μM and 10 μM).

The assay was performed in such a way that approximately 2.5×104 cells/well (100 μL) were treated with 100 μL of all synthetic peptides at different concentrations (100 μM, 10 μM, 1 μM and 0.1 μM) while rTGF-β1 (recombinant antibody) at 100 ng/ml was used as a positive control. After incubating the cells for 12, 24 and 48 hours at 37° C. in 5% CO2, 20 ml of supernatant was transferred to a Greiner plate to which 80 ml of Quanti-blue solution was added. The plate was incubated for 1 hour at 37° C. in 5% CO2 and the production of SEAP (secreted alkaline phosphatase) was detected at 655 nm. All tests were performed in triplicate.

### Example 3—Effects of TGF-β1 Mimetic Peptides in the NFKB Pathway

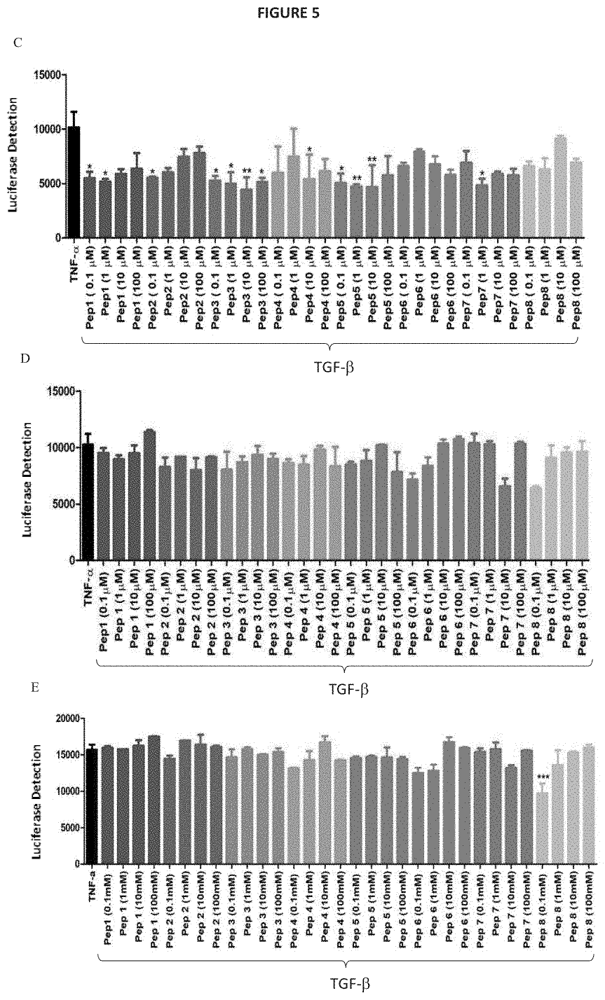

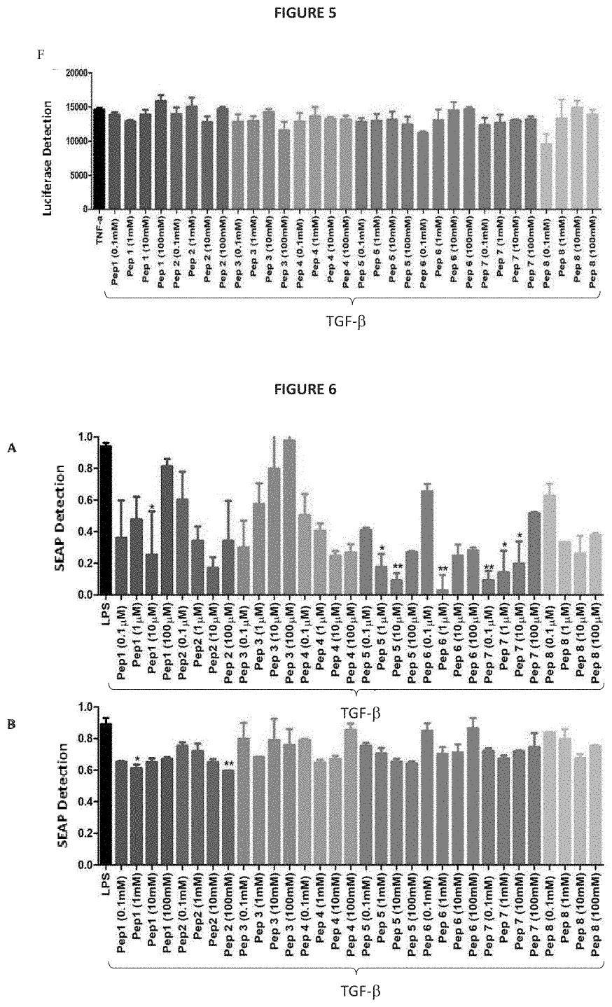

Two different cell treatments were performed using Jurkat cells to analyze NFkB expression after 12, 24 and 48 hours. In the first test, Jurkat cells were treated only with peptides (no pathway activating molecule) at different concentrations to analyze the ability of each peptide to induce NFkB expression (Figure not shown). None of the tested concentrations induced any type of pathway activation. According to this result, cells were pretreated with peptides for 1 hour followed by TNF-α stimulation (FIG. 5A, B, C) and vice versa (pretreated with TNF-α for 1 hour followed by addition of the peptides) (FIG. 5D, E, F).

Peptide pretreatment was not able to reduce NFKB activation after 12 hours of treatment (FIG. 5A); however, when cells were analyzed after 24 hours (FIG. 5B), the peptides were able to decrease the activation of the NFKB pathway compared to the TNF-α control, particularly for some peptides and at specific concentrations we obtained statistically significant results such as: Pep 3 (0.1 μM; 10 μM and 100 μM) (P<0.05; P<0.005 and P<0.05), Pep 4 (0.1 μM; 10 μM and 100 μM) (P<0.05; P<0.005 and P<0.05), Pep 5 (0.1 μM; 1 μM and 100 μM) (P<0.05), Pep 6 (0.1 μM) (P<0.005) and Pep 7 (1 μM and 100 μM) (P<0.05). After 48 hours of treatment (FIG. 5C), the peptides were able to decrease the activation of the NFKB pathway compared to the TNF-α control, and particularly for some peptides and at specific concentrations we obtained statistically significant results such as: Pep 1 (0 0.1 μM and 1 μM) (P<0.05), Pep 2 (0.1 μM) (P<0.05), Pep 3 (0.1 μM; 1 μM; 10 μM and 100 μM) (P<0.05); P<0.05; P<0.005; P<0.05), Pep 4 (10 μM) (P<0.05), Pep 5 (0.1 μM; 1 μM and 10 μM) (P<0.05; P<0.005; P<0.005) and Pep 7 (1 μM) (P<0.05) decreased NFKB expression compared to the TNF-α control.

Finally, some peptides were able to reduce the NFKB pathway when cells were pretreated with TNF-α followed by the addition of the peptide after 12, 24 and/or 48 hours (FIGS. 5D, 5E and 5F). Pep 8 (0.1 μM) was the only peptide to present a statistically significant result, being able to reduce the pathway after 24 hours (P<0.005), when cells were pretreated with TNF-α followed by the addition of the peptide (FIG. 5E).

The assay was carried out in such a way that a total of 3×105 cells/well (100 pl) was pretreated with 100 ml of a composition comprising one of the synthetic peptides in concentrations of 100 μM, 10 μM, 1 μM, and 0.1 μM for 1 hour at 37° C. in 5% CO2, followed by stimulation with TNF-α (200 ng/ml) for 12, 24 and 48 hours. The same stimuli were applied to cells pretreated with TNF-α (200 ng/ml) for 1 hour at 37° C. in 5% CO2 followed by stimulation with peptides at 100 μM, 10 μM, 1 μM and 0.1 μM for 12, 24 and 48 hours. Then, 50 mI of supernatant was transferred to a white plate and Quanti-Luc reagent was used to measure the luciferase product. As a control, the peptides were tested at the same concentrations, without any pathway activating molecule, to investigate whether they could activate the NFkB pathway. TNF-α (200 ng/mL) was used as a positive control and rIL-10 (0.4 ng/mL) as a negative control. a (200 ng/mL).

### Example 4—Action of TGF-β1 Mimetic Peptides in the TLR4 Pathway

Similar to the treatment using Jurkat cells, peptides in compositions with different concentrations were also tested in HEK cells to investigate their ability to interfere with the TLR4 pathway.

To ensure that the peptides could not induce any activation when tested alone (no pathway activation molecule) only the peptides were added into the cell line and SEAP production was measured after 12 hours of treatment (Figure not shown). In fact, peptides at all concentrations did not activate the signaling pathway.

All peptides were able to reduce the activation of the TLR4 pathway, particularly for some peptides at specific concentrations, and we obtained statistically significant results such as: Pep 1 (10 μM) (P<0.05), Pep 5 (1 μM and 10 μM) (P<0.05 and P<0.005), Pep 6 (1 μM) (P<0.005) and Pep 7 (0.1 μM; 1 μM and 10 μM) (P<0.005; P<0.05 and P<0.05) (FIG. 6A). When cells were pretreated with LPS followed by addition of peptides, although all peptides interfered with pathway activation, compared to the positive stimulus, Pep 1 (1 μM) (P<0.05) and Pep 2 (100 μM) (P<0.005) showed a statistically significant result (FIG. 6B).

The assay was performed using HEK-Blue hTLR4 reporter cells (InvivoGen) to investigate the action of peptides in the TLR4 pathway. A total of 2.3×104 cells/well (100 ml) was pretreated with 100 ml of composition comprising at least one of the synthetic peptides at concentrations of 100 μM, 10 μM, 1 μM, and 0.1 μM for 1 hour at 37° C. ° C. in 5% CO2 followed by LPS stimulation at 100 ng/mL for 12 hours. The same stimuli were applied to cells pretreated with LPS at 100 ng/ml for 1 hour at 37° C. in 5% CO2, followed by stimuli with peptides at 100 μM, 10 μM, 1 μM and 0.1 μM for 12 hours. Stimuli were added in Quanti-blue medium and SEAP production was measured at 655 nm. As a control, peptides at the same concentrations were tested alone to investigate whether they could activate the TLR4 pathway and LPS was used as a positive control.

All synthetic peptides described in the present application, with affinity for the TGF-β receptor, exhibited an ability to modulate the autoimmune and inflammatory response, confirming that they can be used in the treatment of chronic or acute inflammatory, allergic and/or auto-inflammatory diseases. In addition, results presented herein demonstrate the potential for association of these peptides, since they presented distinct and, in several cases, complementary responses regarding the concentration and exposure time with the best response. Therefore, they can be used alone or in any combination thereof, in compositions to prepare drugs or immunogenic compositions for the treatment and/or prophylaxis of diseases in which there is an immunological disorder, more particularly in which diseases related to immunological disorders are: chronic or acute inflammatory, allergic and/or autoimmune diseases.

Since all peptides responded, we selected as an example Pep 2-TGF-β1 to show additional data that deepens our knowledge. Such results can be extrapolated to other synthetic peptides with affinity to the TGF-β receptor synthesized and demonstrated in the present patent application.

### Example 5—the Synthesized TGF-B1 Mimetic Peptides have Anti-Inflammatory Properties and Suppress In Vitro IqE-Mediated Basophil Destruction

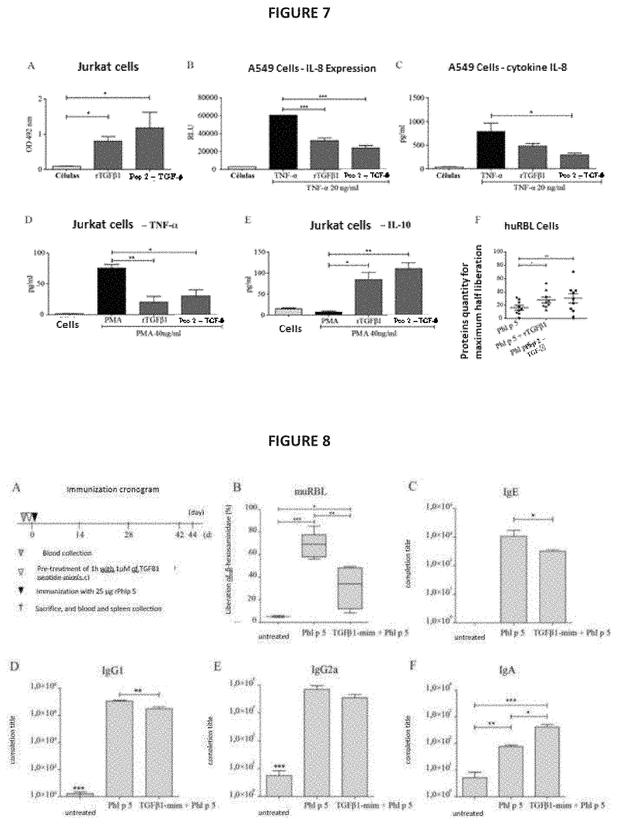

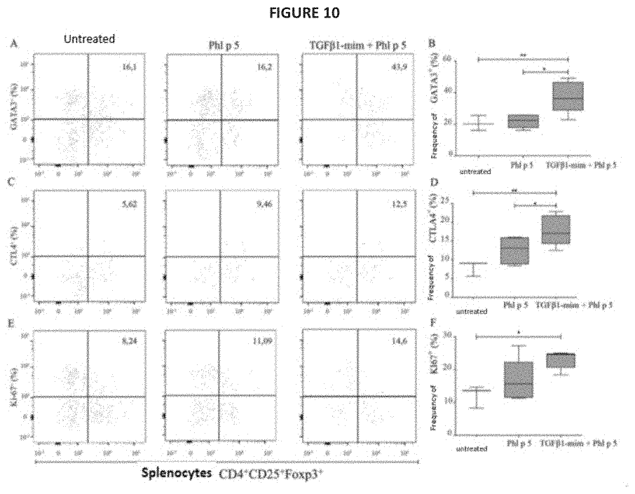

First, we tested the ability of the synthesized peptides to recognize TGF\hat{ }RII on the surface of Jurkat cells, immortalized human T lymphocytes, using ELISA. The recombinant TGFβ (rTGFβ1) was used as a positive control. In the example shown here, Pep 2 had higher levels of reactivity to TGF-βRII than rTGF-β1 (FIG. 7A). To investigate IL-8 cytokine production, we used an immortalized human lung epithelial cell line (A549), which plays an important role in the first defense line to foreign compounds. In TNF-α stimulated A549 cells with a reporter gene for luciferase under the control of the IL-8 promoter, both Pep 2 and tTũRbI decreased IL-8 gene expression (FIG. 7B), whereas only Pep 2 decreased the production/secretion of IL-8 (FIG. 10). In MMA-stimulated Jurkat cells, both the mimetic peptide Pep 2 and rTGFβ significantly decreased TNF-α secretion (FIG. 7D) and increased IL-10 secretion (FIG. 7E). A mediator release assay, based on rat basophilic leukemia (RBL) cells bearing humanized FIERI receptor, was performed to investigate the effect of the synthesized peptides on IgE-mediated degranulation of basophils in an already sensitized system. This sensitive cell-based assay may help to understand the action of the mimetic peptide in an established allergic microenvironment. We found that the amount of Phl p 5 (grass antigen) required to induce half the maximal release in basophils, which were passively sensitized with sera from patients allergic to grass pollen, was significantly higher when the cells were pretreated with Pep 2 (FIG. 7F), indicating down-regulation of degranulation. Such effect was also observed when pretreatment with the rTGFβ, control was performed.

Together, these results indicate that Pep 2-TGF-β1, as well as the other peptides synthesized in this application, are also able to modulate the allergic inflammatory microenvironment in vitro.

### Example 6—Synthesized TGF-β1 Mimetic Peptides Modulate Antibody Response in a Murine Sensitization Model

We then tested the ability of the synthesized TGF-β1 mimetic peptides to modulate the in vivo immune response during allergic sensitization to Phl p 5. We selected as an example Pep 2-TGF-β1 to show the results. Intradermal immunization with the recombinant Phl p5 allergen for 44 days (FIG. 8A) significantly induced the allergen specific total IgE response, as seen from the ability of the sera to cause basophil degranulation in the RBL assay (FIG. 8E and FIG. 8B). RBL cells sensitized with sera from mice treated with 1 μM of Pep 2-TGF-P1 (in FIG. 8A-F represented as TGFβ-mim) had significantly lower levels of degranulation than the Ph; p 5 immunized group. ELISA analysis showed that in the serum of mice sensitized by Phl p5, Pep 2-TGF-β1 suppressed the levels of IgE (FIG. 8C), IgG1 (FIG. 8D) and IgG2a (FIG. 8E), although the latter was not statistically significant. Mice treated with Pep 2-TGF-β1 also showed increased levels of IgA (FIG. 8F). Therefore, Pep 2-TGF-β1 modulated Phl p5-specific antibody responses in mice. This result indicates that the TGF-β1 mimetic peptides synthesized in the present application are also capable of modulating such a response.

### Example 7—Synthesized Peptides Mimetic to TGF-B1 Modulate Cytokine Production in Splenocytes Restimulated with pHl p5

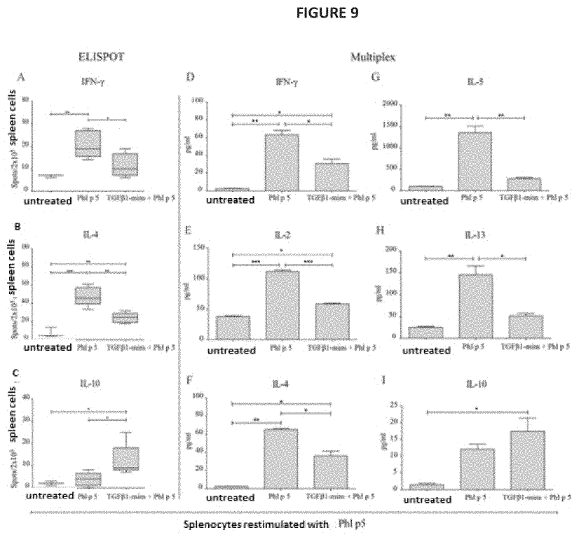

To determine the number of splenic cells producing IFN-g, IL-4 and IL-10 in all groups of mice, an ELISPOT assay was performed. Immunization with Phl p 5 resulted in high induction of IFN-g and IL-4 release in allergen stimulation. In this example, in mice treated with Pep 2-TGF-β1 (in FIG. 9A-I represented as TGF-β-mim), the levels of IFN-g (FIG. 9A) and IL-4 (FIG. 9B), secreted by splenocytes stimulated with Phl p5, were significantly lower, while IL-10 levels were significantly higher (FIG. 9C), than in untreated mice. To investigate the cytokine profile in supernatants from restimulated splenocytes, a Luminex Multiplex cytokine analysis kit was used. The secretion of the cytokines IFN-g (FIG. 9D), IL-2 (FIG. 9E), IL-4 (FIG. 9F), IL-5 (FIG. 9G) and IL-13 (FIG. 9H) was significantly lower, while IL-10 secretion (FIG. 9I) was higher in mice that received Pep 2-TGF-β1 than in untreated mice. Therefore, Pep 2-TGF-β1 downregulated cytokines TH1 and TH2 involved in the immune response to Phl p5 in mice. Furthermore, an increase in IL-10 levels was observed. Such result indicates that the TGF-β1 mimetic peptides synthesized in the present application are also able to downregulate TH1 and TH2 cytokines involved in the immune response to Phl p5 in mice.

### Example 8—Synthesized Peptides Mimetic to TGF-B1 Modulate Treq Cell Production

To investigate the influence of synthesized peptides mimetic to TGF-β1 on Treg induction specific to Phl p5 during allergic sensitization, we analyzed splenocytes from BALB/c mice using flow cytometry. We sought to investigate CD4+CD25+Foxp3+cells expressing GATA3, CTLA4 or KI67, as these factors are necessary for the survival and function of Treg cells. Control and sensitized mice and Phl p5 had comparable levels of Treg cells expressing the factor GATA3 (16.1% and 16.2% of total T cells, respectively), whereas, in the example shown here, mice treated with the Pep 2-TGF-β1 (in FIG. 10A-F represented as TGF-β-mim) had 43.9% (FIG. 10A-B). The percentage of CD4+CD25+Foxp3+T cells expressing the CTLA4 factor in mice without sensitization and sensitized with Phl p 5 was 5.62% and 9.46%, respectively, while in mice treated with Pep 2-TGF-β1 this value was 12.5% (FIG. 10C-D). Although not significantly different when compared to Phl p5-sensitized mice, the percentage of CD4+CD25+Foxp3+cells expressing factor KI67 was enriched in the group of mice treated with Pep 2-TGF-β1 (FIG. 10E-F). Such results showed that Pep 2 promotes the production of Treg cells during sensitization with Phl p 5. This result indicates that the TGF-β1 mimetic peptides synthesized in the present application are also able to promote the production of Treg cells during sensitization with Phl p 5.

### Example 9—Synthesized Peptides Mimetic to TGF-β1 Suppress TH2 Polarization In Vivo

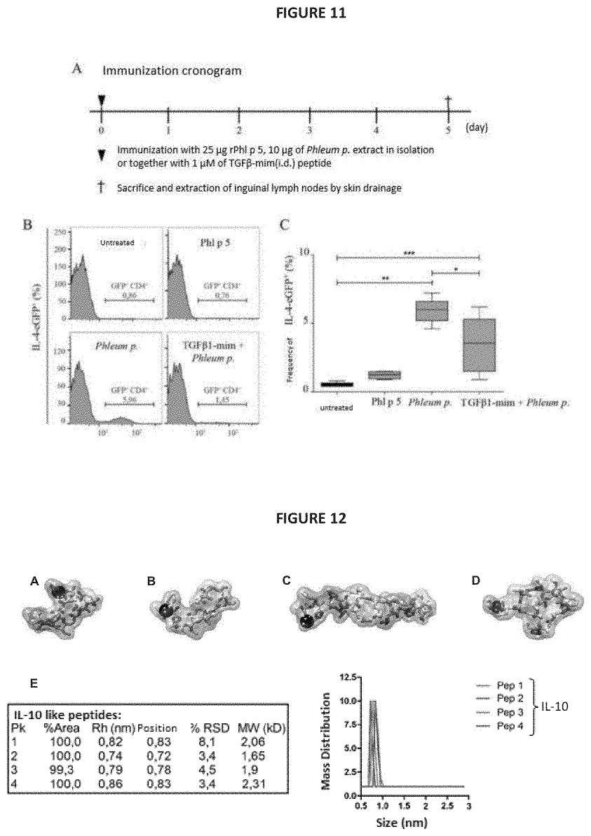

TH2 lymphocytes produce cytokines such as IL-4, IL-5 and IL-13 and lead to the class shift of immunoglobulin production from B cells to IgE, which triggers the allergic reaction. To investigate whether synthesized peptides mimetic to TGF-β1 can modulate TH2 polarization in the presence of grass pollen extract or rPhl p 5, we used an IL-4 reporter mouse. 4get mice express GFP as part of a bicistronic IL-4-IRES-GFP mRNA, allowing the identification of cells expressing IL-4 in situ during an allergen-induced TH2 response. In this experiment, we use skin-draining inguinal lymph node cells to monitor the TH2 response. In the example described here, flow cytometry analysis of GFP+CD4+ lymphocytes isolated from 4get mice 5 days after immunization (FIG. 11A) showed that Pep 2-TGF-β1 (in FIGS. 11A-C represented as TGFβ1-mim), administered over grass pollen extract, suppressed TH2 polarization (FIGS. 11 B-C). Phl p 5 injected alone did not induce the polarization of a TH2 profile in this short-term model. In summary, in this IL-4 mice model, Pep 2 administered in combination with grass pollen extract was able to inhibit TH2 polarization in skin-draining inguinal lymph node cells. Such result indicates that the peptides synthesized in the present application are also able to inhibit TH2 polarization in inguinal lymph node cells that drain the skin.

Synthetic Peptides with Affinity for the IL-10 Receptor

The present patent application describes 4 synthetic peptides with affinity to the Interleukin-10 receptor which comprise or consist of the amino acid sequences: peptide 1-IL-10 (Pep 1-IL-10): SEQ ID 09; peptide 2-IL-10 (Pep 2-IL-10): SEQ ID NO: 10; peptide 3-IL-10 (Pep 3-IL-10) SEQ ID NO: 11; peptide 4-IL-10 (Pep 4-IL-10) SEQ ID NO: 12. Said peptides showed important activity on immunological modulatory and regulatory pathways. Therefore, they can be used in the treatment and/or prophylaxis of diseases related to an immune disorder, such as: chronic or acute inflammatory, allergic and/or autoimmune diseases.

The peptides described in this patent application were selected using the Phage Display (PD) methodology. They were randomly obtained after three rounds of selection using a PhD-7mer and PhD12 random peptide library. Selection was performed using the J774 cell line; for this, a competitive elution with rlL-10 (11-10 recombinant) was adopted to select peptides with affinity to the IL-10 receptor.

A pre-screening was performed on Peripheral Blood Mononuclear Cells (PBMC) using phage supernatant to select phage from the PhD-7mer and PhD12 libraries.

The most reactive phages were purified and tested against PBMC to confirm the ability of selected phages to bind to cell receptors.

Peptides with high affinity for cell receptors were then chemically synthesized following the phage selection manual (Barbas, C. F. et al. Phage Display: a laboratory manual. Cold Spring Harbor Laboratory Press (2001)) and their three-dimensional structures were predicted. All of them were synthesized to present Histidine as the first amino acid residue, and a sequence of three Glycine and a Serine as the last four amino acid residues.

Peptide 1-IL-10 (Pep 1-IL-10). SEQ ID NO 09, has its three-dimensional structure, with disulfide bridges, predicted in FIG. 12A.

Peptide 2-IL-10 (Pep 2-IL-10). SEQ ID NO 10, has its three-dimensional structure, without disulfide bridge, predicted in FIG. 12B.

Peptide 3-IL-10 (Pep 3-IL-10). SEQ ID NO 11, has its three-dimensional structure predicted in FIG. 12C.

Peptide 4-IL-10 (Pep 4-IL-10). SEQ ID NO 12, has its three-dimensional structure predicted in FIG. 12D.

Pep 1-IL-10 and Pep 2-IL-10 were synthesized with an identity or similarity of at least 85% between them. The amino acid residues at positions three (3) and eleven (11) were substituted from Serine in Pep 2 to Cysteine in Pep 1. Therefore, Pep 1-IL-10 is the only peptide that was synthesized with a disulfide bridge, being its three-dimensional structure distinct from the structure of Pep 2-IL-10 and more compact compared to Pep 3-IL-10 and Pep 4-IL-10. In FIGS. 12A to 12D, the black sphere is representative of the C-alpha atom of the first residue, for orientation only, while hydrophilic residues are in blue and the hydrophobic residues in red.

The aggregation behavior of Pep 1-IL-10, Pep 2-IL⋅10, Pep 3-IL-10 and Pep 4-IL-10 peptides in solution was determined by Dynamic Light Scattering (DLS), as can be seen in FIG. 12E. All synthetic peptides were found to be approximately 100% monomeric in solution. The measured hydrodynamic radius of the peptides was similar between Pep 1-IL-10 (0.82 nm), Pep 2-IL-10 (0.74 m), Pep 3-IL-10 (0.79 nm) and Pep 4-IL-10 (0.86 nm).

Once the selected peptides were synthesized, several assays were carried out in order to determine the activity of these peptides. It is noteworthy that in the examples that we will demonstrate below and in the figures attached here, all peptides in general and in different concentrations showed surprising and positive results in modulating the immune, inflammatory and allergic response. However, we will highlight here, throughout the description, just as an example, those results that were statistically more significant. This does not exclude any other results that have not been expressly mentioned in the examples throughout the descriptive report, but which can be directly verified in the figures.

### Example 10—Treatment of Reporter Cells with IL-10 Mimetic Peptides

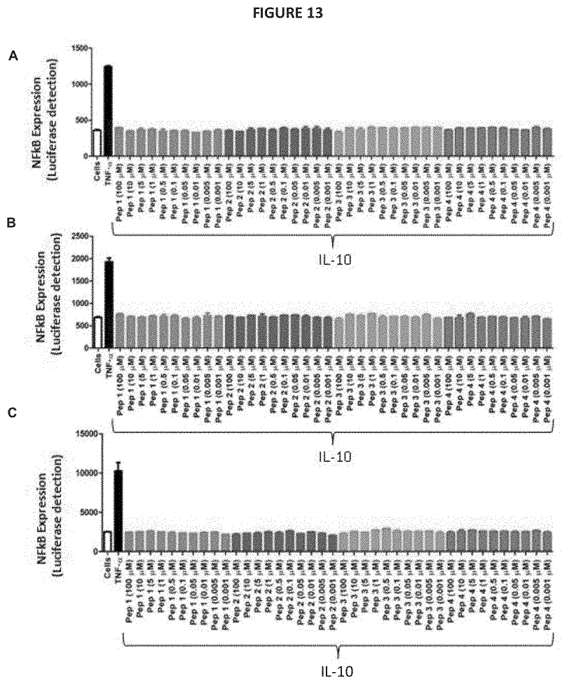

In order to investigate the ability of synthetic peptides with affinity to the Interleukin-10 receptor to interfere in the NFkB, IFN and TLR4 pathways, reporter cell lines were used. To analyze whether the isolated peptides (without activation molecules) could induce any type of activation, the isolated peptides were tested in cell lines during 12, 24 and 48 hours of treatment. In all reporter cells and at all times tested, synthetic peptides with affinity for the IL-10 receptor did not induce any type of activation when they were alone, without any inflammatory environment to induce activation of the NFKB, IFN or TLR4 pathways. As an example, FIG. 13A, FIG. 13B, and FIG. 13C show an analysis of NFkB expression after stimulation with the synthesized IL-10 receptor affinity synthetic peptides described herein. Cells were treated for 12, 24 and 48 hours with the synthesized peptides Pep 1-IL10, Pep 2-IL10, Pep 3-IL10 and Pep 4-IL10, at different concentrations, without activating molecules. Luciferase was then measured after 12 hours of treatment (FIG. 13A), 24 hours of treatment (FIG. 13B) and 48 hours of treatment (FIG. 13C). All tested peptide concentrations were not able to induce any response. TNF-α (200 ng/mL) was used as a positive control.

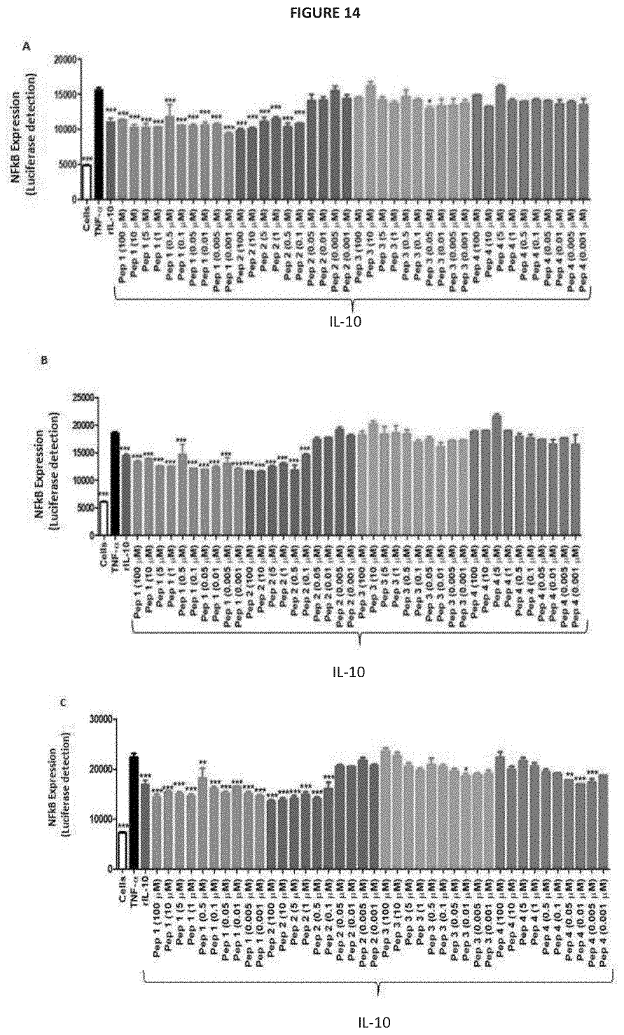

The assay was then carried out using TNF-α as activation molecule not only in the control, but after a pretreatment with the synthesized peptides Pep 1-IL10, Pep 2-IL10, Pep 3-IL10 and Pep 4-IL10, at different concentrations. All peptides were able to modulate NFKB activation after 12, 24 and 48 hours, with different results depending on the concentrations used. Pretreatment with Pep 1-IL10 at all concentrations tested was, with statistical significance, able to decrease NFKB activation after 12, 24 and 48 hours (P<0.0005) of stimulation with TNF-α (FIGS. 14 A, B and C). Pretreatment with Pep 2 had the most statistically significant results at 100 μM, 10 μM, 5 μM, 1 μM, 0.5 μM and 0.1 μM also decreasing NFKB activation, with statistical significance (P<0.0005), after 12, 24 and 48 hours (FIGS. 14 A, B and C). Pretreatment with Pep 3 had the most statistically significant results at 0.05 μM (P<0.05) reducing pathway activation after 12 hours of treatment (FIG. 14A) and at 0.01 μM (P<0.05) after 48 hours (FIG. 14C), despite having presented results in other concentrations and with 24 hours of treatment, as can be seen in FIGS. 14 A, B and C, but with less significance. Pretreatment with Pep 4 had the most statistically significant results at 0.05 μM, 0.01 μM and 0.005 μM, reducing pathway activation after 48 hours of treatment (FIG. 14C). In the assays shown by FIGS. 14 A, B and C, analysis of NFkB expression was performed in Jurkat-Dual reporter cells. Cells were treated for 1 hour with peptides and TNF-α (200 ng/ml) added. Luciferase was measured after 12 hours of treatment (FIG. 14A), 24 hours of treatment (FIG. 14B) and 48 hours of treatment (FIG. 14C).

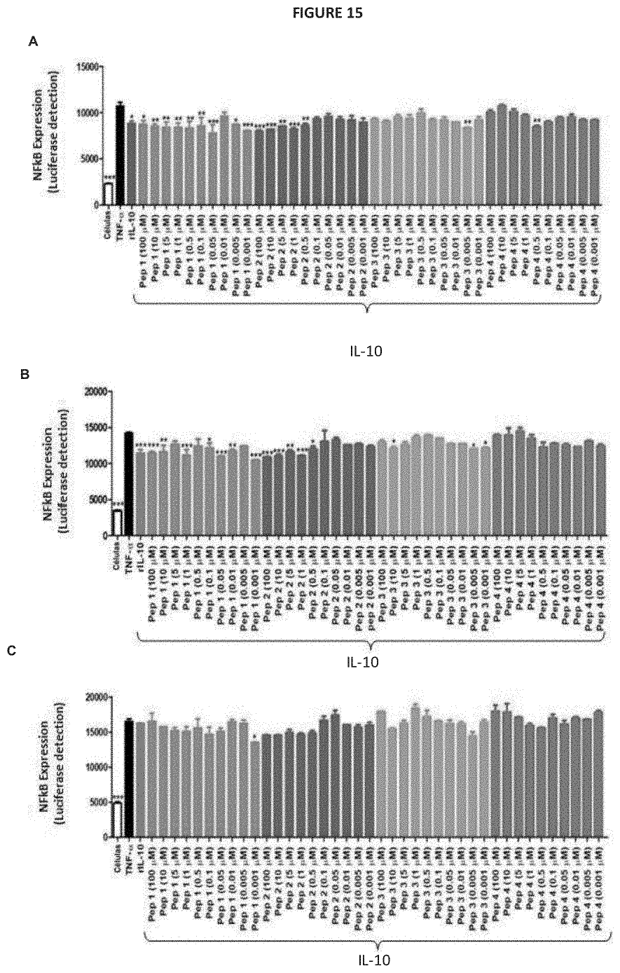

Another assay was then performed in which cells were pretreated with TNF-α and the synthesized peptides Pep 1-IL10, Pep 2-IL10, Pep 3-IL10 and Pep 4-IL10, at different concentrations, were added later. All peptides were able to modulate NFKB activation after 12, 24 and 48 hours, with different results depending on the concentrations used. The most significant results will be described below. For example: Pep 1 at concentrations of 10 μM, 5 μM, 1 μM, 0.5 μM, 0.1 μM (P<0.05), 0.05 μM and 0.001 μM (P<0.0005) decreased pathway activation analyzed after 12 hours of treatment (FIG. 15A). Pep 2 at 100 μM (P<0.0005), 10 μM (P<0.005), 5 μM (P<0.05), 1 μM (P<0.005) and 0.5 μM (P<0.05); Pep 3 at 0.005 μM (P<0.005) and Pep 4 at 0.5 μM (P<0.05) were also able to decrease NFKB signaling after 12 hours of treatment (FIG. 15A). After 24 hours of treatment with Pep 1 at 100 μM (P<0.0005), 10 μM (P<0.005), 1 μM (P<0.0005), 0.1 μM (P<0.05), 0.005 μM (P<0.0005), 0.001 μM (P<0.005) and 0.0001 μM (P<0.0005); Pep 2 at 100 μM and 10 μM (P<0.0005), 5 μM (P<0.005), 1 μM (0.0005) and 0.5 μM (P<0.05); Pep 3 at 10 μM (P<0.05), 0.005 μM (P<0.05) and 0.001 μM (P<0.05), were able to decrease NFKB signaling after 24 hours of treatment (FIG. 15B). After 48 hours of treatment, Pep 1-IL-10 at a concentration of 0.001 μM (P<0.05) was the one that obtained a result with the highest statistical significance in the decrease of NFKB signaling (FIG. 15C). In the assays shown by FIGS. 15 A, B and C, analysis of NFkB expression was performed in Jurkat-Dual reporter cells. Cells were treated for 1 hour with TNF-α (200 ng/ml) and peptides were added. Luciferase was measured after 12 hours of treatment (FIG. 15A), 24 hours of treatment (FIG. 15B) and 48 hours of treatment (FIG. 15C). TNF-α (200 ng/mL) was used as a positive control and rlL-10 (recombinant Interleukin-10) (0.4 ng/mL) as a negative control. *P<0.05; **P<0.005; ***P<0.0005.

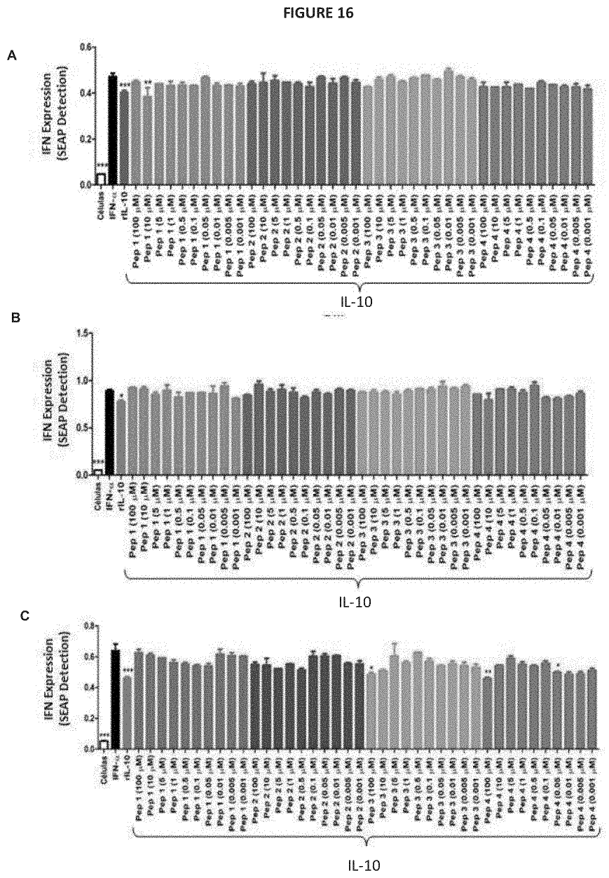

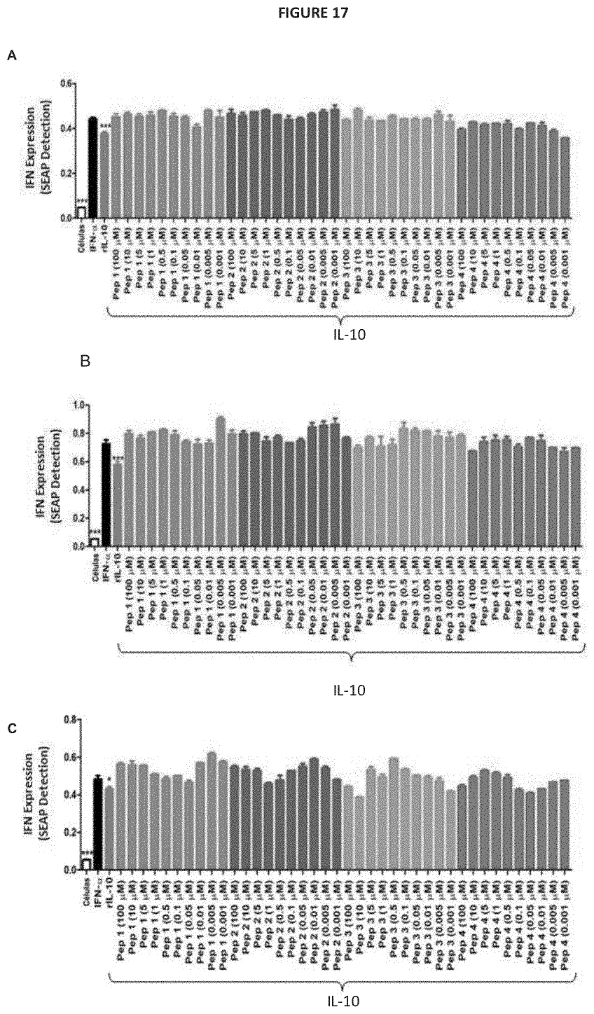

The analysis of the IFN signaling pathway demonstrated that the synthesized peptides Pep 1-IL10, Pep 2-IL10, Pep 3-IL10 and Pep 4-IL10 were able to modulate the activation of the IFN signaling pathway after 12, 24 and 48 hours, with different results depending on the concentrations used. The most significant results will be described below. For example, pathway activation was diminished when Jurkat cells were pretreated with 10 μM Pep 1 (P<0.005) for 12 hours (FIG. 16A); 100 μM Pep 3 (P<0.005) for 48 hours (FIG. 16C); and Pep 4 at 100 μM (P<0.005) and 0.05 μM (P<0.05) for 48 hours (FIG. 16C). When cells were pretreated with IFN, although peptides at some concentrations indicated results, they were not statistically sufficient. Therefore, we cannot confirm whether they were able to interfere with pathway activation (FIGS. 17 A, B and C) in an equal or better way than the negative control (rIL-10). In the assays shown by FIGS. 16 (A, B and C) and FIGS. 17 (A, B and C), analysis of IFN expression was performed in Jurkat-Dual reporter cells. In the assays shown by FIGS. 16 A, B and C, cells were treated for 1 hour with peptides with further addition of IFN-α (104 EU/ml). Secreted Embryonic Alkaline Phosphatase (SEAP) production was measured after 12 hours of treatment (FIG. 16A), 24 hours of treatment (FIG. 16 B) and 48 hours of treatment (FIG. 16 C). In the assays shown by FIGS. 17A, B and C, cells were treated for 1 hour with IFN-α (104 EU/ml) with further addition of the peptides. SEAP production was measured after (A) 12 hours of treatment (B), 24 hours of treatment and (C) 48 hours of treatment. IFN-α (104 EU/ml) was used as a positive control and rlL-10 (0.4 ng/ml) as a negative control. *P<0.05.

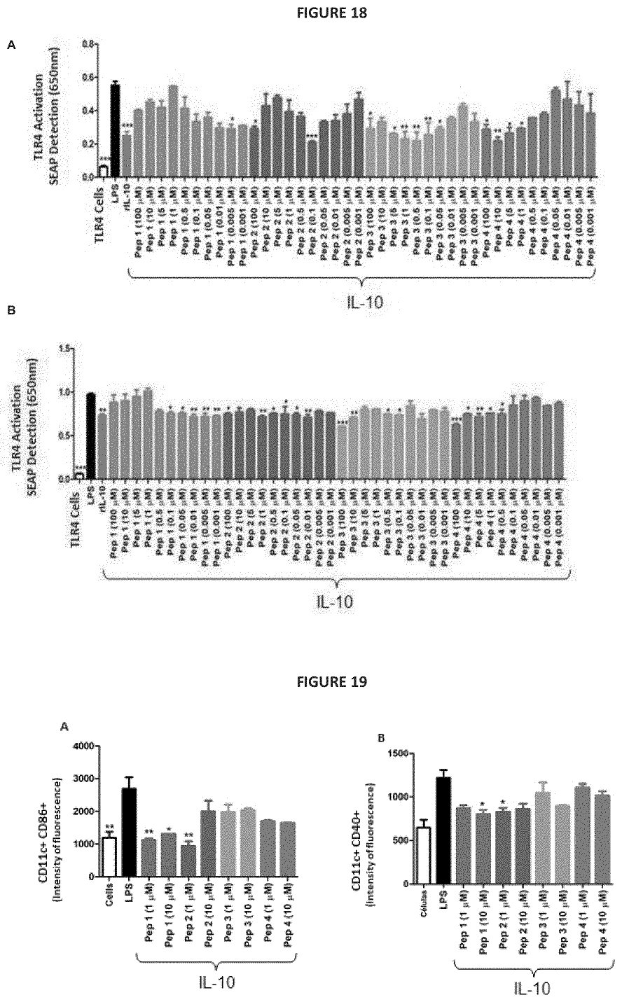

HEK cells were used to investigate the ability of the synthesized peptides Pep 1-IL10, Pep 2-IL10, Pep 3-IL10 and Pep 4-IL10 to modulate the activation of the TLR4 signaling pathway after 12 hours of treatment. All peptides were able to modulate the activation of the TLR4 signaling pathway after 12 hours, with different results depending on the concentrations used. The most significant results will be described below. For example, pretreatment with the peptides demonstrated that Pep 1 at 0.005 μM (P<0.05); Pep 2 at 100 μM (P<0.05), 0.1 μM (P<0.0005); Pep 3 at 100 μM (P<0.05), 5 μM (P<0.05), 1 μM (P<0.005), 0.5 μM (P<0.005), 0.1 μM (P<0.005) and 0.005 μM (P<0.05); and Pep 4 at 100 μM (P<0.05), 10 μM (P<0.005), 5 μM (p<0.05) and 1 μM (P<0.05) significantly reduced pathway activation (FIG. 18A). On the other hand, when cells were pretreated with lipopolysaccharides (LPS) and peptides were added, activation of the TLR4 pathway was significantly reduced when added Pep 1 at 0.1 μM (P<0.05), 0, 05 μM (P<0.05), 0.01 μM (P<0.005), 0.005 μM (P<0.005) and 0.001 μM (P<0.005); Pep2 at 100 μM (P<0.05), 1 μM (P<0.005), 0.5 μM (P<0.05), 0.1 μM (P<0.05), 0.05 μM (P<0.05) and 0.01 μM (P<0.005); Pep 3 at 100 μM (P<0.0005), 10 μM (P<0.005), 0.5 μM (P<0.05) and 0.1 μM (P<0.05); Pep 4 at 100 μM (P<0.0005), 10 μM (P<0.05), 5 μM (P<0.005), 1 μM (P<0.05) and 0.5 μM (P<0.005) (FIG. 18B).

In the assay shown by FIG. 14A cells were treated 1 hour with peptides and LPS (100 ng/ml) was added. SEAP production was measured after 12 hours of treatment. In the assay shown by FIG. 14B cells were treated with LPS (100 ng/ml) for 1 hour and peptides were added. SEAP production was measured after 12 hours of treatment. LPS (100 ng/mL) was used as a positive control and IL-(0.4 ng/mL) as a negative control. *P<0.05; **P<0.005; ***P<0.0005.

### Example 11—Effect of IL-10 Mimetic Peptides on Dendritic and T Cells in an Inflammatory and Allergic Environment

Murine bone marrow dendritic cells were isolated from mice to analyze the effect of synthesized peptides Pep 1-IL-10, Pep 2-IL-10, Pep 3-IL-10 and Pep-4-IL-10, at two concentrations, 1 μM and 10 μM, in the modulation of cell activation. After 24 hours of treatment with a composition comprising at least one of the peptides in the concentration After 24 hours of treatment with a composition comprising at least one of the peptides at the desired concentration, cell analysis showed that all four synthesized peptides showed responses at different levels and were able to interfere with the co-stimulatory molecules present on the surface of dendritic cells, being the Pep 1 and 2 peptides the ones that presented the most significant responses. For example, Pep 1 at 1 μM decreased CD86+ expression (P<0.005), and at 10 μM it also decreased CD86+ expression (P<0.05). Pep 2 at 1 μM reduced the expression of CD86+ and CD40+ on the cell surface (P<0.005; P<0.05, respectively) (FIGS. 19 A and B).

In the assays, CD11c+ CD86 cells (FIG. 19A) and CD11c+ CD40+ cells (FIG. 19B) were used after 24 hours of treatment. LPS (100 ng/mL) was used as a positive control. *P<0.05; **P<0.005.

Since all four synthetic peptides described in the present application, with affinity to the IL-10 receptor, exhibited an ability to modulate the autoimmune and inflammatory response, suggesting that they can be used in the treatment of chronic or acute inflammatory diseases, allergic and/or autoimmune, we selected as an example Pep 1-IL-10 to show the additional data that deepen the knowledge. Furthermore, the results presented here demonstrate the potential for association of these peptides, since they showed distinct and, in many cases, complementary responses regarding concentration and exposure time with better response. Therefore, they can be used alone or in any combination thereof, in compositions to prepare drugs or immunogenic compositions for the treatment and/or prophylaxis of diseases in which there is an immune disorder, more particularly wherein the diseases related to the immune disorder are: chronic or acute inflammatory, allergic and/or autoimmune diseases.

Such results can be extrapolated to other synthetic peptides with affinity to the IL-10 receptor synthesized and demonstrated in the present patent application.

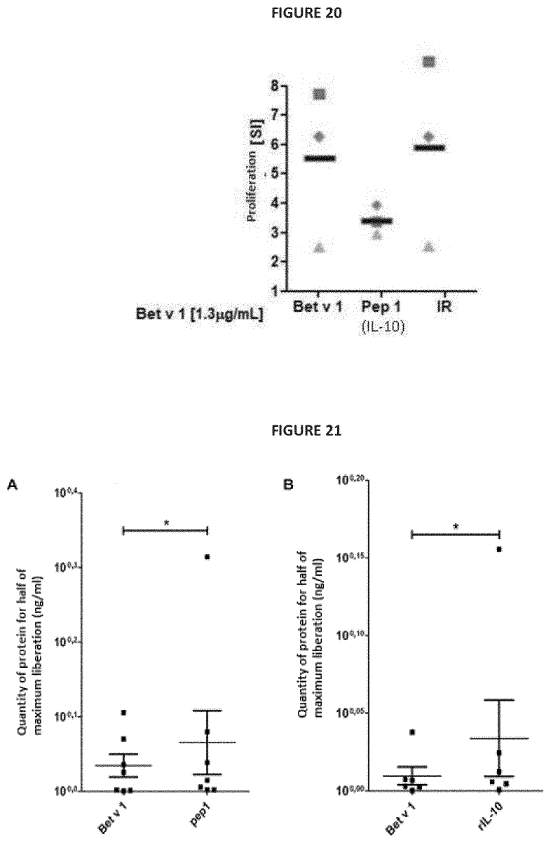

In order to show the ability of synthesized peptides to modulate the activation of dendritic cells, Pep 1 to 10 μM was chosen and the proliferation of T cells from allergic patients was measured after 24 hours of treatment. Pep 1 at this concentration decreased T cell proliferation (P<0.005) (FIG. 20). Cells were treated with Bet v 1 as a positive control and an irrelevant peptide (IR) was used as a negative control.

### Example 12—Action of IL-10 Mimetic Peptides on Basophil Degranulation

The effect of IL-10 mimetic peptides on IgE-mediated release was examined using rat basophilic leukemia cells transfected with the human FIϵRI IgE receptor (hRBL). As an example, we selected as an example Pep 1-IL-10 to show the data obtained. The hRBL cells were sensitized with serum from seven patients allergic to birch pollen. Cells were stimulated with the antigen and its interaction with immobilized IgEs caused a release of b-hexosaminidase.

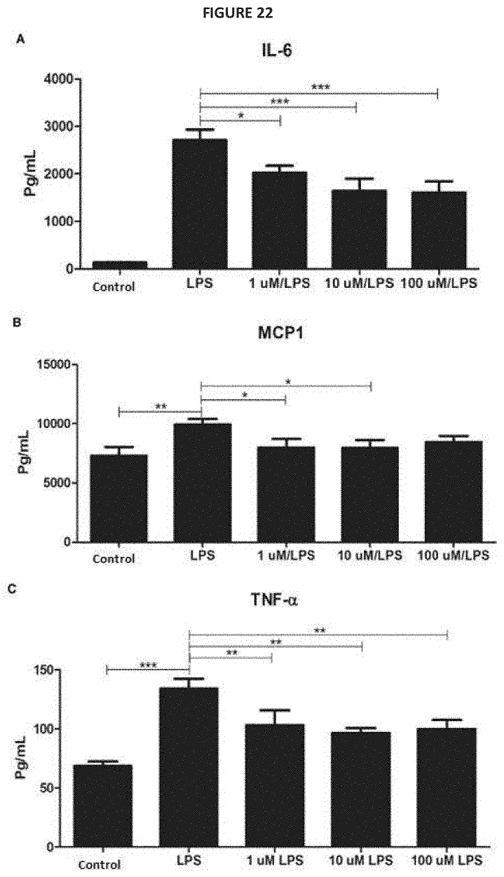

When cells were treated with a composition comprising Pep 1-IL-10 at 1 μM (FIG. 21A) or rIL10 (FIG. 21B), the amount of Bet v 1 needed to induce the same amount of degranulation was higher when compared to cells treated only with Bet vl (P<0.05). Amount of rBet v 1 (ng/mL) required to induce half the release of b-hexosaminidase using serum from seven birch pollen allergic donors after treatment with Pep 1-IL-10 (A) (P<0.05) and rIL10 (B)*P<0.05.

### Example 13—Cytokine Production after Treatment of the J774 Cell Line with IL-10 Mimetic Peptides

After 24 hours of treatment, the levels of IL-6, MCP-1 and TNF-α produced were measured. No interference with the production of IL-10, IFN-g or IL12p70 was observed (data not shown). In the example shown here, cells treated only with the synthetic peptide Pep 1-IL-10, without LPS stimulation to induce an inflammatory response, were unable to induce any response (data not shown). On the other hand, when cells were treated with a composition comprising Pep 1-IL-10 followed by incubation with LPS (1 mg/ml) for 24 hours to induce an inflammatory response, the peptide was able to decrease IL production-6, MCP-1 and TNF-α.

Pep1-IL-10 (1 μM, 10 μM and 100 μM, 1 μM P<0.05 and 10 μM and 100 μM P<0.0005) significantly reduced IL-6 levels (1 μM and 10 μM, P<0.05) (FIG. 22A); MCP-1 (FIG. 22B) and TNF-α (1 μM, 10 μM and 100 μM, P<0.005) (FIG. 22C). Decreases were significant compared to cells treated with LPS alone (positive control). *P<0.05; **P<0.005; ***P<0.0005.

Studies show consistent immunoregulatory actions of peptides mimetic to TGF-β and mimetic to IL-10 synthesized and described herein. In an inflammatory environment, such peptides were able to modulate several important pathways in regulatory actions to suppress an inflammatory response. In the case of TGF-β, mimetic peptides, it positively modulated IL-10. We therefore investigated the combination of TGF-β and IL-10 mimetic peptides.

### The Combination of TGF-β1 and IL-10 Mimetic Peptides

### Example 14—the Combination of TGF-β1 and IL-10 Mimetic Peptides Suppresses Basophil Degranulation

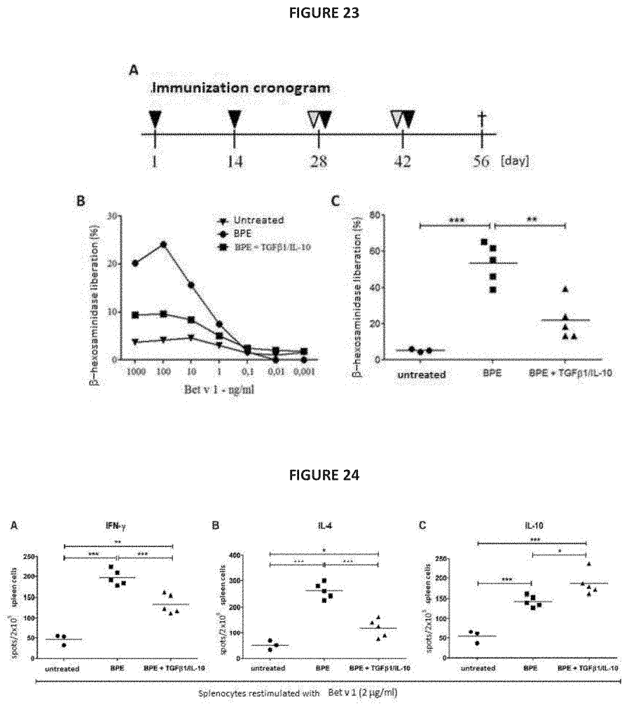

The ability of synthesized TGF-β1 and IL-10 mimetic peptides to modulate the specific IgE immune response to Bet v 1 was investigated by mediator release assays using RBL-2H3 cells passively sensitized with IgE antibodies from mice immunized with extract of birch pollen. Bet v 1 was able to induce IgE production, as seen in the high levels of basophil degranulation in the group of mice immunized with birch pollen extract (FIG. 23B-C). On the other hand, mice immunized with birch pollen extract in combination with TGF-β1 and IL-10 mimetic peptides, in the example presented here, we use the combination of Pep 2-TGF-β1 with Pep 1-IL-10, and they had significantly lower levels of basophil degranulation compared to the control group (P<0.01).

The combination of TGF-β1 and IL-10 mimetic peptides suppresses IgE-specific basophil degranulation. FIG. 23 A shows the scheme of animal immunizations. FIG. 23B shows the titration curve for the mediator release assay performed with sera from all groups, showing that mice treated with the synthesized peptides had substantially lower levels of basophil degranulation. FIG. 23C shows the mediator release assay performed with individual sera reinforcing the action of peptides in the downregulation of IgE-specific basophil degranulation. **P<0.01, ***P<0.001.

For immunization, female BALB/c mice (6-10 weeks old) were used in this study. The immunization scheme was performed as described in FIG. 23A. To induce allergen specific IgE response, five mice were immunized intradermally (i.d.) with 125 pg of birch pollen extract diluted in PBS. Five mice were immunized (i.d.) with 125 pg of birch pollen extract in combination with TGF-β and IL-10 mimetic peptides diluted to 1 μM in PBS each. In the example shown here, we use the combination of Pep 2-TGF-β1 with Pep 1-IL-10. Three mice constituted the control group. Immunizations were performed on days 1, 14, 28 and 42, and mice were sacrificed on day 56. To investigate the induction of the specific IgE response to Bet v 1, blood samples were collected from the saphenous vein on days 28 and 42 after the first immunization.

### Example 15—the Combination of TGFB1 and IL-10 Mimetic Peptides Modulates Cytokine Production

The ability of TGF-β1 and IL-10 mimetic peptides to interfere in the modulation of the number of IFN-g, IL-4 and IL-10 producing spleen cells in all groups of mice was investigated by ELISPOT. Immunization with birch pollen extract resulted in high induction of production of IFN-g (P<0.001) and IL-4 (P<0.001) after stimulation with Bet v 1. Mice treated with peptides, in this example with the combination of Pep 2-TGF-β1 with Pep 1-IL-10, had significantly lower levels of IFN-g (P<0.001; FIG. 24A) and IL-4 (P<0.001; FIG. 24B) secreted by splenocytes restimulated by Bet v 1, while IL-10 levels were significantly higher (P<0.05; FIG. 24C) when compared to untreated mice.

Splenocytes restimulated with Bet v 1 and peptides produced significantly lower levels of IFN-g (FIG. 24A) and IL-4 (FIG. 24B), and significantly higher levels of IL-10 production (FIG. 24C) in comparison to untreated group. *P<0.05; **P<0.01; ***P<0.001.

For this experiment, spleens were aseptically removed immediately after mice were sacrificed, lysed and centrifuged for 5 min in 1 ml of minimal essential culture medium (MEM). Lysis of erythrocytes was performed by adding 5 ml of lysis buffer (150 μM NH 4 Cl, 10 μM KFIC03, 0.1 μM Na2EDTA in ddFI20, pH 7.2) for 5 min at room temperature. White blood cells were separated by centrifugation at 300×g for 5 min at room temperature. Cells were resuspended in culture medium (MEM, 1% (v/v) of heat-inactivated fetal bovine serum, 2 μM L-glutamine, 100 U/ml penicillin, 100 U/ml streptomycin, 20 μM HEPES, 1 μM sodium pyruvate, 2 M 2-mercaptoethanol and 1*non-essential amino acids) and used in subsequent experiments.

For detection of IFN-g, IL-4 and IL-10 after stimulation of splenocytes with Bet v 1; MultiScreen filter plates were activated with 70% ethanol for 10 min, washed three times with PBS, and 2 pg/ml of anti-mouse IFN-g, anti-mouse IL-4 or anti-mouse IL-10 was added during night at 4° C. in a humid chamber. Plates were washed three times with PBS and incubated for 2 h with blocking solution (culture medium supplemented with 5% fetal bovine serum) in a humid chamber at RT. Splenocytes were diluted to a density of 2×105 in culture medium, in the presence of Bet v 1 diluted to 20 pg/ml and incubated for 48 hours at 37° C. and 5% CO2. After three washes with PBS supplemented with 0.1% Tween-20; detection antibodies IFN-g, IL-4 and IL-10 were added to the plates and incubated for 2 h at room temperature. After three washes, HRP-conjugated streptavidin was added to the plates and incubated for 1 h at room temperature. After four washes, TMB substrate was added to each plate for 5 min before stopping by washing with ddhteO. Immunospot counting was performed using ImageJ software.

### Example 16—Analysis of Splenocytes and the Ability of TGFB1 and IL-10 Mimetic Peptides to Modulate Cytokine Production with Pretreatment or in Conjunction with Allergen

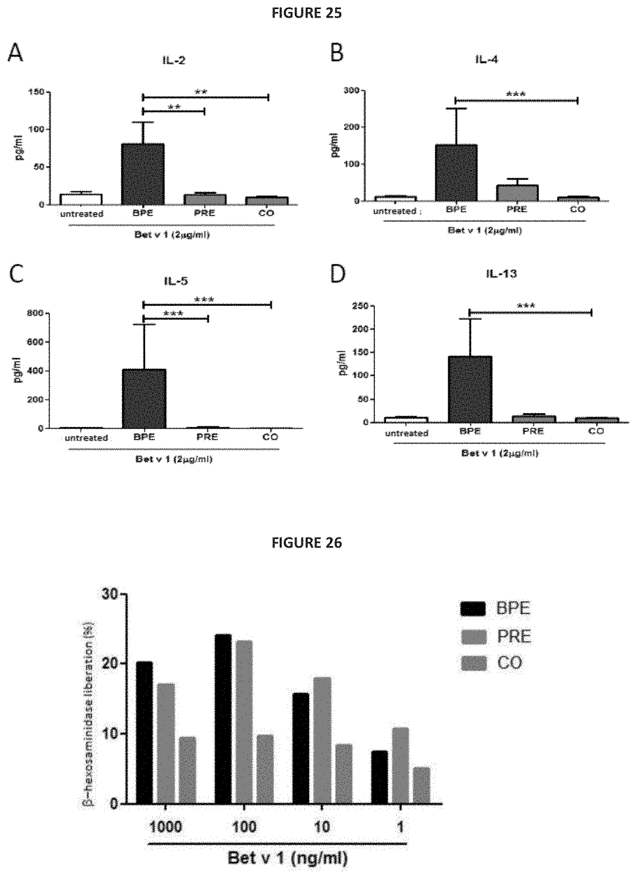

The ability of TGF-β and IL-10 mimetic peptides to modulate cytokine production was investigated using splenocytes and serum from mice immunized with only birch pollen extract (125 pg) (results were identified in FIGS. 25A to 25D as: BPE); pre-immunized for 24 hours with peptide mimetics to TGF-β and IL-10 (1 μM/animal) followed by immunization with birch pollen extract (125 pg) (results were identified in FIGS. 25A to 25D as: PRE); or immunized with the mixture of TGF-β+peptide IL-10+birch pollen extract (results were identified in FIGS. 25A to 25D as: CO). Splenocytes were isolated from mice and the production of IL-4, IL-5, IL-2 and IL-13 was analyzed by Multiplex after challenge with Bet v 1 (2 pg/ml). The mice that were pretreated (PRE) with the combination of TGF-β1 and IL-10 mimetic peptides (Pep 2-TGF-β1+Pep 1-IL-10), showed a significant decrease in the production of all cytokines analyzed. However, surprisingly, mice in the CO group, that is, those treated with the combination of TGF-β1 and IL-10 mimetic peptides, together with the allergen of interest (birch pollen extract), showed results slightly better compared to the group that was pretreated (PRE) with the peptide combination alone. This result indicates that, in vivo, the use of a composition comprising a combination of TGF-β1 and IL-10 mimetic peptides described in the present invention can act synergistically in modulating an immune response. More than that, the results still indicate a synergistic effect not only between the mimetic peptides but also when the allergen was added in the treatment composition. The results were enhanced when the allergen of interest was added together with the combination of mimetic peptides.

### Example 17—Analysis of the Ability of TGF-β1 and IL-10 Mimetic Peptides to Modulate Basophil Degranulation with Pretreatment or in Conjunction with Allergen

The ability of TGF-β and IL-10 mimetic peptides to modulate basophil degranulation was investigated using RBL-2H3 cells sensitized with serum from mice immunized only with birch pollen extract (125 pg) (results were identified in FIG. 26 as: BRE); pre-immunized for 24 hours with the combination of TGF-β and IL-10 mimetic peptides (1 μM/animal) followed by immunization with birch pollen extract (125 pg) (results were identified in FIG. 26 as: PRE); or immunized with the mixture TGF-β+peptide IL-10+birch pollen extract (results were identified in FIG. 26 as: CO). Degranulation was induced by the addition of Bet v 1 (1000 ng/ml, 100 ng/ml, 10 ng/ml and 1 ng/ml). The mice in the CO group, surprisingly, had substantially lower levels of degranulation compared to the other two groups. Suggesting that, in vivo, the combination of peptides, together with the allergen of interest, was able to reduce the production of allergen specific IgE antibodies.

### Analysis of the Results and Embodiments of the Invention

The present patent application demonstrates the ability of new synthesized peptides mimetic to TGF-β1 and mimetic to IL-10 to modulate the response of several important pathways in the regulation of an autoimmune and/or inflammatory process.

In addition, although the potential of the new synthesized peptides described in this patent application in modulating an allergic or immune inflammatory response has already become clear, we deepened the studies and showed the regulatory role of synthesized peptides in modulating an allergic inflammatory response. The peptides were able to modulate basophil degranulation after antigen stimulation, to modulate TH1 and TH2 profile responses through the modulation of cytokines and antibodies, acted in the differentiation of induced Treg cells and affected other important cellular events that promote the exacerbation of the allergic or inflammatory microenvironment.

Allergen-specific immune modulation is an essential process to prevent the development of allergy, wherein IgE plays a key role. Thus, inhibition of IgE production is a crucial event to suppress allergic reactions. In our study, TGF-β and IL-10 mimetic peptides efficiently inhibited basophil degranulation. The ability of these two cytokines to modify the production of IgE-like antibodies in relation to non-inflammatory antibody isotypes has already been reported. However, we demonstrate that the new synthesized peptides described in the present application, which are small molecules, and which can mimic the action of specific large proteins that are able to induce the same or better response as expected with the natural molecule or compared to other synthetic molecules already described. Furthermore, we concluded that there is a surprising synergistic effect with the combination of TGF-β and IL-10 mimetic peptides described in the present invention. Additionally, even more surprising was the synergistic effect when the combination of peptides was associated with the allergen of interest. Therefore, we conclude that the treatment with the combination of TGF-β and IL-10 mimetic peptides had a synergistic effect in modulating an allergic or immune inflammatory response, as well as being able, when in association with an allergen of interest, to significantly reduce the basophil degranulation.

As IFN-g is a cytokine that contributes directly and indirectly to the differentiation of the Th1 profile, while IL-4 is a cytokine that promotes the differentiation of the Th2 profile, the downregulation of these two cytokines indicates a modulation of Th1 or Th2 polarization, essential event to control the inflammatory/allergic response. IL-4 is known to induce IgE class switching in mice (27), thus it may help to explain our previous result, showing that the peptides suppressed IgE-mediated degranulation of basophils. We hypothesize that inhibition of IL-4 consequently suppresses IgE production. On the other hand, the upregulation of IL-10 indicates that the peptides had an anti-inflammatory action, since this cytokine plays a crucial and often essential role in the prevention of inflammatory pathologies. Therefore, we conclude that treatment with TGF-β and IL-10 mimetic peptides modulated the production of cytokines that play a critical role in controlling the immune response.

Furthermore, what was observed in this study, and which is unexpected, was the fact that mice immunization with the combination of mimetic peptides to TGF-β and IL-10, when performed together with the antigen of interest (in the example herein, the birch pollen extract) has resulted in an improved effect both in modulating the allergic response (suppression of basophil degranulation) and in modulating the production of inflammatory cytokines.

Therefore, the present patent application describes synthetic peptides with affinity to the TGF-β receptor and synthetic peptides with affinity to IL-10, to be used in combination of at least one TGF-β mimetic peptide with at least one IL-10 mimetic peptide, in a pharmaceutical composition for use in the treatment and/or prophylaxis of diseases related to immunological disorder, more particularly, chronic or acute inflammatory and/or allergic, and/or autoimmune diseases.

The present application also describes pharmaceutical compositions comprising at least one TGF-β mimic peptide with at least one IL-10 mimetic peptide, plus at least one pharmaceutically acceptable carrier or compound. More particularly the composition may further comprise as a pharmaceutically acceptable compound an allergen of interest. The pharmaceutical composition is for use in the treatment and/or prophylaxis of diseases related to immunological disorder, more particularly, chronic or acute inflammatory and/or allergic, and/or autoimmune diseases.

The present application also describes the use of the synthetic peptides or pharmaceutical compositions described herein, for the preparation of a medicament or immunogenic composition for use in the treatment and/or prophylaxis of diseases related to immunological disorder, more particularly, chronic, or acute inflammatory diseases and/or allergic, and/or autoimmune.

Additionally, the present application also describes methods of treatment and/or prophylaxis of diseases related to immunological disorder which comprises administering to a subject a pharmaceutically effective dose of at least one recombinant peptide with affinity for the TGF-β receptor and at least one peptide recombinant with affinity to the Interleukin-10 receptor, or a pharmaceutical composition comprising the combination thereof. More particularly since it also comprises jointly administering an allergen of interest.

Although the present patent application has described the subject matter of the present invention with a certain degree of detail by way of illustration and example for purposes of clarity and understanding, it will be evident that certain changes and modifications may be practiced within the scope of the appended claims.

The examples described in this report are not limiting, allowing a person skilled in the art to change some aspects or components of the present invention, equivalent to the synthetic peptides, compositions or uses described herein, without departing from the scope of the present invention.

1. A pharmaceutical composition, comprising:
at least one recombinant peptide with affinity for the TGF-B receptor;
at least one recombinant peptide with affinity for the Interleukin-10 receptor; and
at least one pharmaceutically acceptable vehicle, carrier, or compound.

2. The pharmaceutical composition according to claim 1, wherein the recombinant peptide with affinity for the TGF-B receptor comprises or consists of the amino acid sequence selected from the group consisting of: SEQ ID NO: 01, SEQ ID NO: 02, SEQ ID NO: 03, SEQ ID NO: 04, SEQ ID NO: 05, SEQ ID NO: 06, SEQ ID NO: 07 and SEQ ID NO: 08, or an amino acid sequence having at least 85% identity or similarity to one of the sequences selected from the group consisting of: SEQ ID NO: 01, SEQ ID NO: 02, SEQ ID NO: 03, SEQ ID NO: 04, SEQ ID NO: 05, SEQ ID NO: 06, SEQ ID NO: 07 and SEQ ID NO: 08.

3. The pharmaceutical composition according to claim 1, wherein the recombinant peptide with affinity to the Interleukin-10 receptor comprises or consists of the amino acid sequence selected from the group consisting of: SEQ ID NO: 01, SEQ ID NO: 02, SEQ ID NO: 03 and SEQ ID NO: 04 or a sequence having at least 85% identity or similarity to one of the amino acid sequences selected from the group consisting of: SEQ ID NO: 01, SEQ ID NO: 02, SEQ ID NO: 03 and SEQ ID NO: 04.

4. The pharmaceutical composition according to claim 1, wherein the pharmaceutically acceptable compound is selected from an allergen of interest.

5. The pharmaceutical composition according to claim 1, wherein it is intended for use in the treatment and/or prophylaxis of diseases related to immune disorders.

6. The pharmaceutical composition according to claim 5, wherein the diseases related to the immune disorder are: chronic or acute inflammatory, allergic and/or autoimmune diseases.

7. A method for treating and/or preventing an immunological disease comprising the step of administering the pharmaceutical composition of claim 1 to a subject in need thereof.

8-9. (canceled)

10. A method of treatment and/or prophylaxis of diseases related to an immune disorder, the method comprising the step of administering a dose to an individual a pharmaceutically effective of at least one recombinant peptide with affinity for the TGF-β receptor and at least a recombinant peptide with affinity for the Interleukin-10 receptor, ora the pharmaceutical composition claim 1.

11. The method according to claim 10, further comprising the step of administering together an allergen of interest.

12. The method according to claim 10, wherein the recombinant peptide with affinity for the TGF-B receptor comprises or consists of the amino acid sequence selected from the group consisting of: SEQ ID NO: 01, SEQ ID NO: 02, SEQ ID NO: 03, SEQ ID NO: 04, SEQ ID NO: 05, SEQ ID NO: 06, SEQ ID NO: 07 and SEQ ID NO: 08, or an amino acid sequence having at least 85% identity or similarity to one of the sequences selected from the group consisting of: SEQ ID NO: 01, SEQ ID NO: 02, SEQ ID NO: 03, SEQ ID NO: 04, SEQ ID NO: 05, SEQ ID NO: 06, SEQ ID NO: 07 and SEQ ID NO: 08.

13. The method according to claim 10, wherein the recombinant peptide with affinity to the Interleukin-10 receptor comprises or consists of the amino acid sequence selected from the group consisting of: SEQ ID NO: 01, SEQ ID NO: 02, SEQ ID NO: 03 and SEQ ID NO: 04 or a sequence having at least 85% identity or similarity to one of the amino acid sequences selected from the group consisting of: SEQ ID NO: 01, SEQ ID NO: 02, SEQ ID NO: 03 and SEQ ID NO: 04.

14. The method of claim 13, wherein the diseases related to the immune disorder are chronic or acute inflammatory, allergic and/or autoimmune diseases.

15. The pharmaceutical composition according to claim 2, wherein the pharmaceutically acceptable compound is selected from an allergen of interest.

16. The pharmaceutical composition according to claim 3, wherein the pharmaceutically acceptable compound is selected from an allergen of interest.

17. The pharmaceutical composition according to claim 2, wherein it is intended for use in the treatment and/or prophylaxis of diseases related to immune disorders.

18. The pharmaceutical composition according to claim 3, wherein it is intended for use in the treatment and/or prophylaxis of diseases related to immune disorders.

19. The pharmaceutical composition according to claim 4, wherein it is intended for use in the treatment and/or prophylaxis of diseases related to immune disorders.


SEQUENCE LISTING (12 sequences)

SEQ ID NO: 1 - protein, 15 aa, Artificial Sequence
  Synthetic peptide
HACFLPASGLCGGGS

SEQ ID NO: 2 - protein, 15 aa, Artificial Sequence
  Synthetic peptide
HACESPLKRQCGGGS

SEQ ID NO: 3 - protein, 15 aa, Artificial Sequence
  Synthetic peptide
HASESPLKRQSGGGS

SEQ ID NO: 4 - protein, 15 aa, Artificial Sequence
  Synthetic peptide
HACKLQRSEPCGGGS

SEQ ID NO: 5 - protein, 15 aa, Artificial Sequence
  Synthetic peptide
HASFLPASGLSGGGS

SEQ ID NO: 6 - protein, 15 aa, Artificial Sequence
  Synthetic peptide
HASKLQRSEPSGGGS

SEQ ID NO: 7 - protein, 15 aa, Artificial Sequence
  Synthetic peptide
HASTIASTLHSGGGS

SEQ ID NO: 8 - protein, 15 aa, Artificial Sequence
  Synthetic peptide
HASTLPSNTHSGGGS

SEQ ID NO: 9 - protein, 15 aa, Artificial Sequence
  Synthetic peptide
HACSAVIKSSCGGGS

SEQ ID NO: 10 - protein, 15 aa, Artificial Sequence
  Synthetic peptide
HASSAVIKSSSGGGS

SEQ ID NO: 11 - protein, 16 aa, Artificial Sequence
  Synthetic peptide
HCFDTQRMEFRMPPGG

SEQ ID NO: 12 - protein, 16 aa, Artificial Sequence
  Synthetic peptide
HCMTGSRATFPSAPGG